SEQUENCE LISTING (28 sequences)

SEQ ID NO: 1 - DNA, 495 bp, Pneumonia virus
gtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtacc

SEQ ID NO: 2 - DNA, 495 bp, Pneumonia virus; identical to SEQ ID NO: 1

SEQ ID NO: 3 - DNA, 495 bp, Pneumonia virus; identical to SEQ ID NO: 1

SEQ ID NO: 4 - DNA, 495 bp, Pneumonia virus; identical to SEQ ID NO: 1

SEQ ID NO: 5 - DNA, 495 bp, Coronavirus
gtgagatggtcatgtgtggcggctcactatatgttaaaccaggtggaacatcatccggtgatgctacaactgcttatgctaatagtgtctttaacatttgtcaagctgttacagccaatgtaaatgcactcctttcaactgatggtaacaagatagctgacaagtacgtccgcaatctacaacacaggctttatgagtgtctctatagaaatagggatgttgatcatgaatttgtggatgagttttacgcttacctgcgtaaacatttctctatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacc

SEQ ID NO: 6 - DNA, 495 bp, Coronavirus
gtgagatggtcatgtgtggcggctcactatatgttaaaccaggtggaacatcatccggtgatgctacaactgcttatgctaatagtgtctttaacatttgtcaagctgttacagccaatgtaaatgcactcctttcaactgatggtaacaagatagctgacaagtacgtccgcaatctacaacacaggctttatgagtgtctctatagaaatagggatgttgatcatgaattcgtggatgagttttacgcttacctgcgtaaacatttctctatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacc

SEQ ID NO: 7 - DNA, 495 bp, Coronavirus
gtgagatggtcatgtgtggcggctcactatatgttaaaccaggtggaacatcatcaggtgatgctacaactgcttatgctaatagtgtctttaacatttgtcaagctgttacagccaatgtaaatgcacttctttcaactgatggtaacaagatagctgacaagtatgtccgcaatctacaacacaggctttatgagtgtctctatagaaatagggatattgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacc

SEQ ID NO: 8 - DNA, 495 bp, Coronavirus
gtgagatggtcatgtgtggcggctcactatatgttaaaccaggtggaacatcatccggtgatgctacaactgcttatgctaatagtgtctttaacatttgtcaagctgttacagccaatgtaaatgcacttctttcgactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaacaatgtgttcatgtctgaggcaaaatgttggactgagactgaccttactaaaggacctcacgaattttgctcacagcatactatgctagttaaacaaggagatgattacgtgtacc

SEQ ID NO: 9 - DNA, 495 bp, Coronavirus
gtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggcaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaacaatgtgttcatgtctgaggcaaaatgttggactgagactgaccttactaaaggacctcacgaattttgctcacagcatacaatgctagttaaacaaggagatgattacgtgtacc

SEQ ID NO: 10 - DNA, 495 bp, Coronavirus
gtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaacaatgtgttcatgtctgaggcaaaatgttggactgagactgaccttactaaaggacctcacgaattttgctcacagcatacaatgctagttaaacaaggagatgattacgtgtacc

SEQ ID NO: 11 - DNA, 495 bp, Coronavirus; identical to SEQ ID NO: 10

SEQ ID NO: 12 - DNA, 495 bp, Coronavirus
gtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacc

SEQ ID NO: 13 - DNA, 495 bp, Coronavirus; identical to SEQ ID NO: 12

SEQ ID NO: 14 - DNA, 495 bp, Coronavirus; identical to SEQ ID NO: 12

SEQ ID NO: 15 - DNA, 495 bp, Coronavirus; identical to SEQ ID NO: 12

SEQ ID NO: 16 - DNA, 495 bp, Coronavirus; identical to SEQ ID NO: 12

SEQ ID NO: 17 - DNA, 495 bp, Coronavirus; identical to SEQ ID NO: 12

SEQ ID NO: 18 - DNA, 494 bp, Coronavirus
gtgaaatggtcatgtgtggcggttcactatatgttaagcctggtggcacctcatctggagatgccactactgcttatgctaatagtgttttcaacatatgtcaggctgtgactgctaatgtgaatgcactcttatctactgatggtaataaaattgccgacaagtatgtccgcaatctacaacacagactttatgagtgtctctataggaatagagatgttgatcatgaattcgtgggagagttttacgcttatttgcgtaaacatttttccatgatgattctttcagatgatgctgttgtgtgctataatagcaactatgcagcacaaggtctagtggctagcattaagaacttcaaagcagttctttactaccaaaataatgttttcatgtctgaagcaaaatgttggactgagactgaccttactaaaggacctcatgagttttgctctcagcatacaatgctagttaaacaaggagatgattatgtgtac

SEQ ID NO: 19 - DNA, 495 bp, Coronavirus
gtgaaatggttatgtgtggcggttcactctatgtgaaaccaggcggtacatcttcaggagatgccaccactgcttatgctaatagtgtctttaacatttgtcaagctgttacagctaatgttaatgcacttttgtctactgatggtaataaaattgctgacaagtatgtccgcaatttacaacatagactttatgaatgtctctatagaaatagagacgttgatcatgaatttgtagaagaattttacgcttatttgcgtaaacacttttctatgatgattctctctgatgatgctgttgtttgctataatagcaactatgcagctcaaggtttagtagctagcattaagaactttaaagcagttctttattatcaaaacaatgtttttatgtctgaggcaaaatgctggactgagaccgaccttactaaaggacctcatgaattttgctctcagcatacaatgctagttaaacaaggagatgattacgtgtacc

SEQ ID NO: 20 - DNA, 495 bp, Coronavirus
gtgaaatggtcatgtgtggcggttcactttatgtgaagccagggggtacatcttccggagatgccaccactgcttatgctaacagtgtttttaacatttgtcaggctgttacagctaatgttaatgcacttttgtctactgatggtaataagattgctgacaagtatgtccgcactttacaacatagactttatgaatgtctctataggaatagagatgttgatcatgaatttgtagaagaattttacgcttatttgcgtaaacacttttctatgatgattctttctgatgacgctgtcgtttgctataatagcaactatgcagctcaaggtttagtggctagcatcaagaactttaaagcagttctttattaccaaaataatgtttttatgtctgaggcaaaatgctggactgagacagaccttactaaaggacctcatgaattttgctctcagcatacaatgctagttaaacaaggagatgattatgtgtacc

SEQ ID NO: 21 - DNA, 21281 bp, Coronavirus
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtcggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 22 - DNA, 24 bp, Artificial Sequence
  Primer
cttggctattcagttgttgctatc

SEQ ID NO: 23 - DNA, 22 bp, Artificial Sequence
  Primer
gtgtttttaacatttgtcaagc

SEQ ID NO: 24 - DNA, 22 bp, Artificial Sequence
  Primer
gtgtttttaacatttgtcaagc

SEQ ID NO: 25 - DNA, 18 bp, Artificial Sequence
  Primer
tgtttacgcaaatatgcg

SEQ ID NO: 26 - DNA, 18 bp, Artificial Sequence
  Primer
gtggcggytcacthtatg

SEQ ID NO: 27 - DNA, 18 bp, Artificial Sequence
  Primer
gctagcyactaraccttg

SEQ ID NO: 28 - DNA, 18 bp, Artificial Sequence
  Primer
tgaggtcctttagtaagg

(19) United States
(12) Patent Application Publication
(10) Pub. No.: US 2023/0295746 A1
(43) Pub. Date: Sep. 21, 2023

(54) A DETECTION METHOD

(71) Applicant: Microbio Pty Ltd, Westlake, Queensland (AU)

(72) Inventor: Flavia Huygens, Westlake, QLD (AU)

(73) Assignee: Microbio Pty Ltd, Westlake, Queensland (AU)

(21) Appl. No.: 18/018,523

(22) PCT Filed: Jul. 27, 2021
(86) PCT No.: PCT/AU2021/050813

§ 371 Date: Jan. 27, 2023

(30) Foreign Application Priority Data
Jul. 27, 2020 (AU) 2020902627

(51) Int. Cl. C12Q 1/70; C12Q 1/6844

(52) U.S. Cl. CPC C12Q 1/701; C12Q 1/6844; C12Q 2600/156

(57) ABSTRACT

The present invention relates to a method for detecting severe acute respiratory syndrome coronavirus 2 (SARS-CoV-2) in a sample, said method including the step of analysing at least a portion of a viral RNA-dependent RNA polymerase (RdRP) gene or gene product from the sample for the presence or absence of at least one single nucleotide polymorphism (SNP), wherein SARS-CoV-2 is detected in the sample based on the presence or absence of the at least one SNP.

### CROSS REFERENCE TO RELATED APPLICATIONS

This is the U.S. National Stage of International Application No. PCT/AU2021/050813, filed Jul. 27, 2021, which was published in English under PCT Article 21(2), which in turn claims the benefit of Australian Patent Application No. 2020902627, filed Jul. 27, 2020, which is incorporated herein in its entirety.

### TECHNICAL FIELD

This invention relates to detection methods. More particularly, this invention relates to methods and agents for detecting severe acute respiratory syndrome coronavirus 2 (SARS-CoV-2) in samples, such as biological samples. The methods and agents are based on the use or detection of polymorphisms within the viral RNA-dependent RNA polymerase (RdRP) gene, such as by high resolution melt analysis.

### BACKGROUND

Coronavirus disease 2019 (COVID-19) is an infectious viral disease caused by severe acute respiratory syndrome coronavirus 2 (SARS-CoV-2). The first cases of this new viral disease were identified in December 2019 in Wuhan, China, which has now escalated into an ongoing global pandemic.

Current testing for COVID-19 aims to detect the causative virus, SARS-CoV-2, or an immune response to SARS-CoV-2. The two main types of SARS-CoV-2 tests are: nucleic acid detection tests using reverse transcriptase-polymerase chain reaction (RT-PCR) to detect SARS-CoV-2 viral RNA; and serological tests to detect IgM and/or IgG antibodies against the SARS-CoV-2 virus.

Nucleic acid/PCR tests are currently considered to be more clinically sensitive than serology assays for detecting early COVID-19 infections, because they directly detect viral RNA. Notwithstanding this, there remains a need for rapid, reliable and cost effective techniques for the accurate detection of SARS-CoV-2 in samples, such as biological samples obtained from human subjects potentially infected with the virus.

### SUMMARY

The present invention is predicated in part on high conservation of the viral RdRP gene between coronavirus strains, including various SARS-CoV-2 strains or isolates, and also in part on the discovery of multiple single nucleotide polymorphisms (SNPs) within the RdRP gene and oligonucleotide or primer sequences that are specific to SARS-CoV-2 that may be useful in the detection of SARS-CoV-2, in a sample.

In a first aspect, the invention provides a method for detecting severe acute respiratory syndrome coronavirus 2 (SARS-CoV-2) in a sample, said method including the step of analysing at least a portion of a viral RNA-dependent RNA polymerase (RdRP) gene or gene product from the sample for the presence or absence of at least one single nucleotide polymorphism (SNP),

wherein the at least one SNP in the at least a portion of the viral RdRP gene or gene product is at a position corresponding to at least one of positions 56, 137, 146, 233, 272 and 380 of the nucleic acid sequence set forth in any one of SEQ ID NOs: 1-4;

wherein SARS-CoV-2 is detected in the sample based on the presence or absence of the at least one SNP.

In some embodiments, the at least one SNP in the at least a portion of the viral RdRP gene or gene product is at a position corresponding to at least one of positions 137, 146 and 233 of the nucleic acid sequence set forth in any one of SEQ ID NOs: 1-4.

Suitably, the step of analysing comprises determining the presence or the absence of the at least one SNP using high resolution melt analysis, 5′ nuclease digestion, molecular beacons, oligonucleotide ligation, microarray, restriction fragment length polymorphism, antibody detection methods, direct sequencing or any combination thereof. In particular embodiments, the step of analysing comprises determining the presence or the absence of the at least one SNP using at least partly high resolution melt analysis.

Suitably, the step of analysing comprises amplifying a nucleic acid of the viral RdRP gene or gene product from the sample. In various embodiments, amplifying the nucleic acid of the viral RdRP gene or gene product utilizes at least in part reverse transcriptase-polymerase chain reaction (RT-PCR) and may include the further steps of obtaining or isolating RNA from the sample and reverse transcribing the RNA to obtain complementary DNA (cDNA).

Suitably, the step of amplifying the nucleic acid includes using one or more primers that hybridize to (e.g., are complementary or substantially complementary to) a portion of the conserved region of the RdRP gene or gene product that includes one or more SNPs described herein. More particularly, the step of amplifying the nucleic acid can include using one or more primers that hybridize to respective portions of the RdRP gene or gene product that include at least one SNP, wherein the at least one SNP is at a position corresponding to at least one of positions 56, 137, 146, 233, 272 and 380 of the nucleic acid sequence set forth in any one of SEQ ID NOs: 1-4.

In certain embodiments, the step of amplifying the nucleic acid of the viral RdRP gene or gene product includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:25, a nucleotide sequence complementary thereto or a fragment or variant thereof. In these embodiments, the at least one SNP may be at a position corresponding to at least one of positions 137, 146 and 233 of the nucleic acid sequence set forth in any one of SEQ ID NOs: 1-4.

In other embodiments, the step of amplifying the nucleic acid of the viral RdRP gene or gene product includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:25, a nucleotide sequence complementary thereto or a fragment or variant thereof. In these embodiments, the at least one SNP may be at a position corresponding to at least one of positions 56, 137, 146 and 233 of the nucleic acid sequence set forth in any one of SEQ ID NOs: 1-4.

In various embodiments, the step of amplifying the nucleic acid of the viral RdRP gene or gene product includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:27, a nucleotide sequence complementary thereto or a fragment or variant thereof. In these embodiments, the at least one SNP may be at a position corresponding to at least one of positions 137, 146, 233 and 272 of the nucleic acid sequence set forth in any one of SEQ ID NOs: 1-4.

In some embodiments, the step of amplifying the nucleic acid of the viral RdRP gene or gene product includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof. In these embodiments, the at least one SNP may be at a position corresponding to at least one of positions 137, 146, 233, 272 and 380 of the nucleic acid sequence set forth in any one of SEQ ID NOs: 1-4.

In particular embodiments, the step of amplifying the nucleic acid of the viral RdRP gene or gene product includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof. In these embodiments, the at least one SNP may be at a position corresponding to at least one of positions 56, 137, 146, 233, 272 and 380 of the nucleic acid sequence set forth in any one of SEQ ID NOs: 1-4.

In a second aspect, the invention resides in a method for detecting severe acute respiratory syndrome coronavirus 2 (SARS-CoV-2) in a sample, said method including the steps of:
(a) amplifying a nucleic acid from a conserved region of a RNA-dependent RNA polymerase (RdRP) gene or gene product of SARS-CoV-2; and
(b) detecting the presence or absence of the nucleic acid amplified in step (a).

Suitably, the conserved region of the RdRP gene or gene product comprises, consists of or consists essentially of a nucleotide sequence or portion thereof set forth in any one of SEQ ID NOs:1-4.

In particular embodiments, the nucleic acid amplified in step (a) includes at least one SNP, wherein the at least one SNP is at a position corresponding to at least one of positions 56, 137, 146, 233, 272 and 380 of the nucleic acid sequence set forth in any one of SEQ ID NOs: 1-4. More particularly, the at least one SNP is suitably at a position corresponding to at least one of positions 137, 146 and 233 of the nucleic acid sequence set forth in any one of SEQ ID NOs: 1-4. In other embodiments, the at least one SNP is at a position corresponding to at least one of positions 56, 137, 146 and 233 of the nucleic acid sequence set forth in any one of SEQ ID NOs: 1-4. In various embodiments, the at least one SNP is at a position corresponding to at least one of positions 137, 146, 233 and 272 of the nucleic acid sequence set forth in any one of SEQ ID NOs: 1-4. In some embodiments, the at least one SNP is at a position corresponding to at least one of positions 137, 146, 233, 272 and 380 of the nucleic acid sequence set forth in any one of SEQ ID NOs: 1-4.

Suitably, the step of amplifying the nucleic acid includes using one or more primers that hybridize to (e.g., are complementary or substantially complementary to) a portion of the conserved region of the RdRP gene or gene product that includes one or more SNPs described herein. More particularly, the step of amplifying the nucleic acid can include using one or more primers that hybridize to respective portions of the conserved region of the RdRP gene or gene product that include at least one SNP, wherein the at least one SNP is at a position corresponding to at least one of positions 56, 137, 146, 233, 272 and 380 of the nucleic acid sequence set forth in any one of SEQ ID NOs: 1-4.

In some embodiments, the step of amplifying the nucleic acid includes using a forward primer comprising, consisting of or consisting essentially of a nucleotide sequence set forth in SEQ ID NO:24, a nucleotide sequence complementary thereto or a fragment or variant thereof and/or a reverse primer comprising, consisting of or consisting essentially of a nucleotide sequence set forth in SEQ ID NO:25, a nucleotide sequence complementary thereto or a fragment or variant thereof.

In these embodiments, the at least one SNP may be at a position corresponding to at least one of positions 137, 146 and 233 of the nucleic acid sequence set forth in any one of SEQ ID NOs: 1-4.

In other embodiments, the step of amplifying the nucleic acid includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:25, a nucleotide sequence complementary thereto or a fragment or variant thereof. In these embodiments, the at least one SNP may be at a position corresponding to at least one of positions 56, 137, 146 and 233 of the nucleic acid sequence set forth in any one of SEQ ID NOs: 1-4.

In various embodiments, the step of amplifying the nucleic acid includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:27, a nucleotide sequence complementary thereto or a fragment or variant thereof. In these embodiments, the at least one SNP may be at a position corresponding to at least one of positions 137, 146, 233 and 272 of the nucleic acid sequence set forth in any one of SEQ ID NOs: 1-4.

In some embodiments, the step of amplifying the nucleic acid includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof. In these embodiments, the at least one SNP may be at a position corresponding to at least one of positions 137, 146, 233, 272 and 380 of the nucleic acid sequence set forth in any one of SEQ ID NOs: 1-4.

In particular embodiments, the step of amplifying the nucleic acid includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof. In these embodiments, the at least one SNP may be at a position corresponding to at least one of positions 56, 137, 146, 233, 272 and 380 of the nucleic acid sequence set forth in any one of SEQ ID NOs: 1-4.

In certain embodiments, the presence or absence of the nucleic acid is detected at least in part using high resolution melt analysis.

In various embodiments, further including the step of analysing at least a portion of the nucleic acid amplified in step (a) for the presence or absence of at least one SNP, wherein the at least one SNP is at a position corresponding to at least one of positions 56, 137, 146, 233, 272 and 380 of the RdRP gene or gene product set forth in any one of SEQ ID NOs: 1-4. In particular embodiments, the at least one SNP is at a position corresponding to at least one of positions 137, 146 and 233 of the nucleic acid sequence set forth in any one of SEQ ID NOs: 1-4. In other embodiments, the at least one SNP is at a position corresponding to at least one of positions 56, 137, 146 and 233 of the nucleic acid sequence set forth in any one of SEQ ID NOs: 1-4. In various embodiments, the at least one SNP is at a position corresponding to at least one of positions 137, 146, 233 and 272 of the nucleic acid sequence set forth in any one of SEQ ID NOs: 1-4. In some embodiments, the at least one SNP is at a position corresponding to at least one of positions 137, 146, 233, 272 and 380 of the nucleic acid sequence set forth in any one of SEQ ID NOs: 1-4.

Amplifying the nucleic acid of the RdRP gene or gene product suitably utilizes RT-PCR and can include the further steps of obtaining RNA from the sample and reverse transcribing the RNA to obtain cDNA.

Referring to the aforementioned aspects, the sample suitably is a biological sample, such as cells, blood, serum, plasma, saliva, cerebrospinal fluid, urine, stool, sputum, and nasopharyngeal aspirates or swabs, obtained from a subject, including human subjects. Accordingly, the methods of the first and second aspects may be used for identifying subjects infected with SARS-CoV-2.

In some embodiments of the aforementioned aspects, the methods further include the step of detecting replication of SARS-CoV-2 in the sample.

In a third aspect, the invention relates to an isolated probe, tool or reagent capable of detecting SARS-CoV-2 in a sample, wherein the probe, tool or reagent is capable of binding, detecting or identifying the presence or absence of at least one SNP in at least a portion of a viral RdRP gene or gene product,

wherein the at least one SNP in the at least a portion of the viral RdRP gene or gene product is at a position corresponding to at least one of positions 56, 137, 146, 233, 272 and 380 of the nucleic acid sequence set forth in any one of SEQ ID NOs: 1-4.

In some embodiments, the probe, tool or reagent comprises an oligonucleotide or primer comprising, consisting of or consisting essentially of a nucleic acid sequence as set forth in at least one of SEQ ID NOs: 24 to 28, a nucleotide sequence complementary thereto or a fragment or variant thereof.

In a fourth aspect, the invention provides an isolated oligonucleotide, primer or nucleic acid molecule comprising, consisting of or consisting essentially of a nucleic acid sequence as set forth in any one of SEQ ID NOs: 24 to 28, a nucleotide sequence complementary thereto or a fragment or variant thereof.

In a fifth aspect, the invention resides in an array comprising: the isolated probe, tool or reagent of the third aspect; and/or the isolated oligonucleotide of the fourth aspect.

In a sixth aspect, the invention relates to a biochip comprising a solid substrate and the isolated probe, tool or reagent of the third aspect; and/or the isolated oligonucleotide of the fourth aspect.

In a seventh aspect, the invention provides a kit or assay for detecting SARS-CoV-2 in a sample, said kit or assay comprising: the isolated probe, tool or reagent of the third aspect; the isolated oligonucleotide of the fourth aspect; the array of the fifth aspect; and/or the biochip of the sixth aspect.

In some embodiments, the kit or assay comprises a pair of oligonucleotides, wherein at least one of the pair of oligonucleotides comprises, consists of or consists essentially of the nucleic acid sequence as set forth in any one of SEQ ID NOs:24 to 28.

In particular embodiments, the kit includes instructions for use, such as instructions for using the pair of oligonucleotides in RT-PCR and high resolution melt analysis.

Suitably, the isolated probe, tool or reagent of the third aspect, the isolated oligonucleotide of the fourth aspect, the array of the fifth aspect, the biochip of the sixth aspect or the kit or assay of the seventh aspect, is for use in the method of the first and/or second aspects.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

By “consist essentially of” is meant in this context that the nucleic acids described herein have one, two or no more than three nucleic acid residues in addition to the recited nucleic acid sequence. The additional nucleic acid residues may occur at the 5′ and/or 3′ ends of the recited nucleic acid sequence, although without limitation thereto.

The indefinite articles ‘a’ and ‘an’ are used here to refer to or encompass singular or plural elements or features and should not be taken as meaning or defining “one” or a “single” element or feature. For example, “a” cell includes one cell, one or more cells and a plurality of cells.

As generally used herein “about” refers to a tolerance or variation in a stated value or amount that does not appreciably or substantially affect function, activity or efficacy. Typically, the tolerance or variation is no more than 10%, 5%, 3%, 2%, or 1% above or below a stated value or amount.

The reference to any prior art in this specification is not, and should not be taken as an acknowledgement or any form of suggestion that the prior art forms part of the common general knowledge.

### BRIEF DESCRIPTION OF THE FIGURES

FIGS. 1A-1F. Whole genome of Wuhan SARS-CoV-2 strain. Exemplary forward and reverse primers are in bold text and boxed. SEQ ID NO: 21 is shown.

FIGS. 2A-C. Differentiation of SARS-CoV-2 strains (bold text and boxed) from other SARS coronaviruses. Identification of SNPs and primer sequences (bold text) in Clustal omega alignment of a conserved region of the RdRP gene of 20 coronavirus strains. The sequences shown are SEQ ID NO:1 (MN988713.1:15431-15925), SEQ ID NO:2 (MN985325.1:15431-15925), SEQ ID NO:3 (MN938384.1:15399-15893), SEQ ID NO:4 (NC_045512.2:15431-15925), SEQ ID NO:5 (KJ473816.1:15126-15620), SEQ ID NO:6 (MK211376.1:15360-15854), SEQ ID NO:7 (KY417146.1:15361-15855), SEQ ID NO:8 (FJ882929.1:15340-15834), SEQ ID NO:9 (FJ882959.1:15354-15848), SEQ ID NO:10 (JF292915.1:15321-15815), SEQ ID NO:11 (KF514407.1:15341-15835), SEQ ID NO:12 (AH013709.2:14120-14614), SEQ ID NO:13 (JX163924.1:15321-15815), SEQ ID NO:14 (JN854286.1:15361-15855), SEQ ID NO:15 (FJ882963.1:15342-15836), SEQ ID NO:16 (EU371563.1:15361-15855), SEQ ID NO:17 (DQ898174.1:15361-15855), SEQ ID NO:18 (KY352407.1:15287-15780), SEQ ID NO:19 (GU190215.1:15260-15754), and SEQ ID NO:20 (MG975784.1:400-894).

FIGS. 3A-B. Identification of SARS-CoV-2 minimum SNPs.

FIG. 4. RT-PCR results for SARS-CoV-2 detection test using a tenfold serial dilution of SARS-CoV-2 total RNA extracted from cell culture. Normalised fluorescence is shown on the Y axis and number of cycles on the X axis.

FIG. 5. High Resolution Melt curve results for SARS-CoV-2 detection test using tenfold serial dilution of SARS-CoV-2 total RNA extracted from cell culture. The change in fluorescence (dF/dT) is shown on the Y axis and temperature is shown on the X axis.

FIG. 6. Exemplary negative and positive HRM curves for the detection of SARS-CoV-2.
(a) shows the results when:
   Internal control: NEGATIVE Valid PCR: no*
   CoV-2: no result
      Possible contribution factors to the failure of the procedure are poor RNA integrity, RNA input concentration outside recommended range and/or presence of PCR inhibitors in sample;
(b) shows the results when:
   Internal control: POSITIVE Valid PCR: yes
   CoV-2: NEGATIVE
(c) shows the results when:
   Internal control: POSITIVE Valid PCR: yes
   CoV-2: POSITIVE

FIG. 7. Instrument panel showing one-step Reverse-Transcriptase Real-time PCR run profile, showing the cycling parameters utilized to identify and differentiate non-replicating SARS-CoV-2 from other SARS-CoV strains.

FIG. 8. Melt curve for Bio-Rad CFX96 real-time PCR machine.

FIG. 9. Melt curve for MIC (Biomolecular Systems) real-time PCR machine.

FIG. 10. Melt curve for QuantStudio 5 (Applied Biosystems) real-time PCR machine.

FIG. 11. Melt curve for RotorGeneQ (Qiagen) real-time PCR machine.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜45,056 bytes), which was created on Jan. 16, 2023, which is incorporated by reference herein.

### BRIEF DESCRIPTION OF THE SEQUENCES

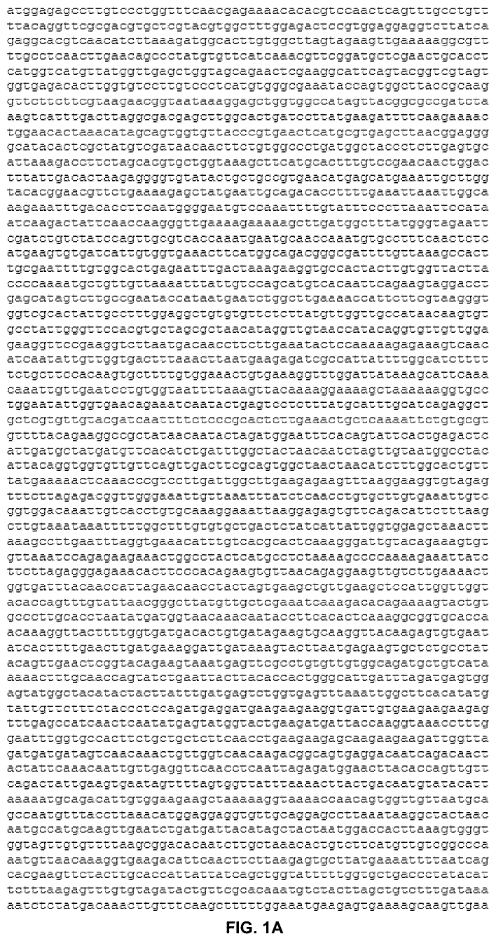

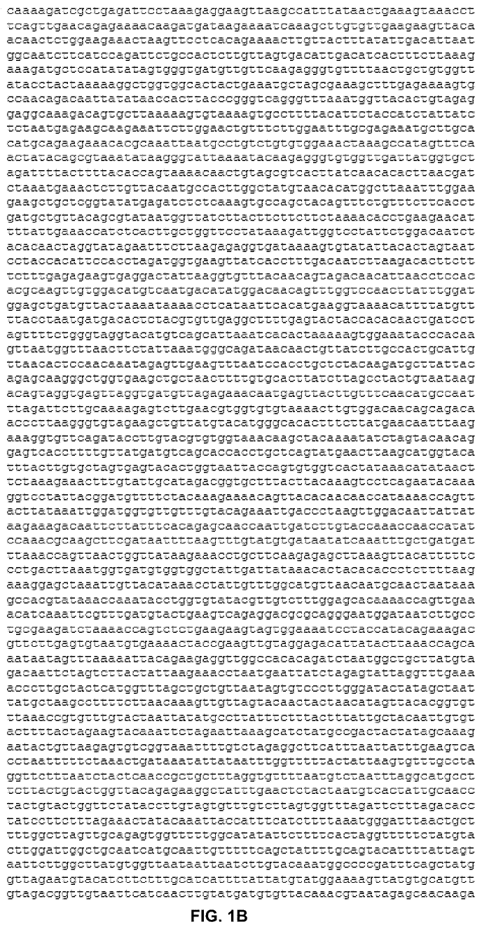

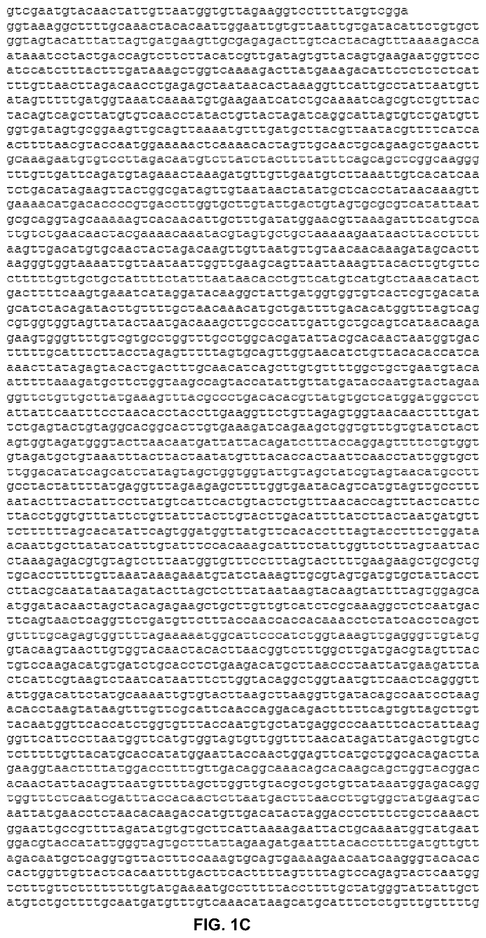

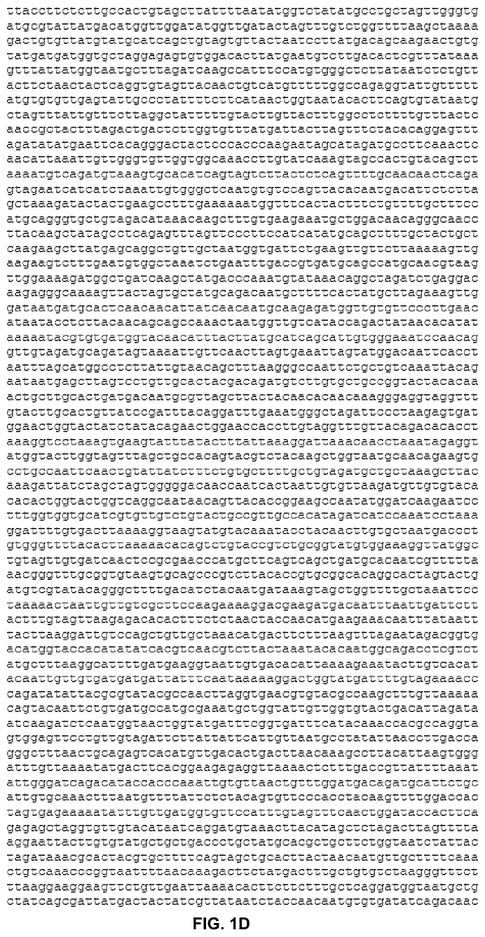

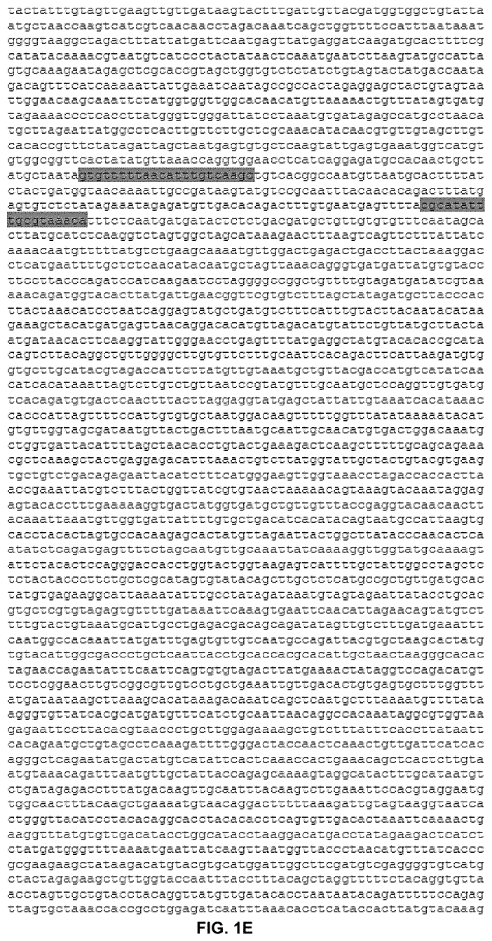

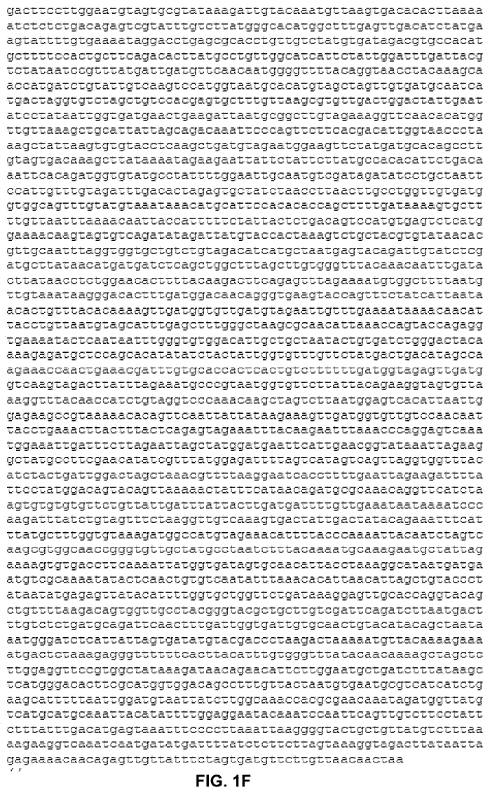

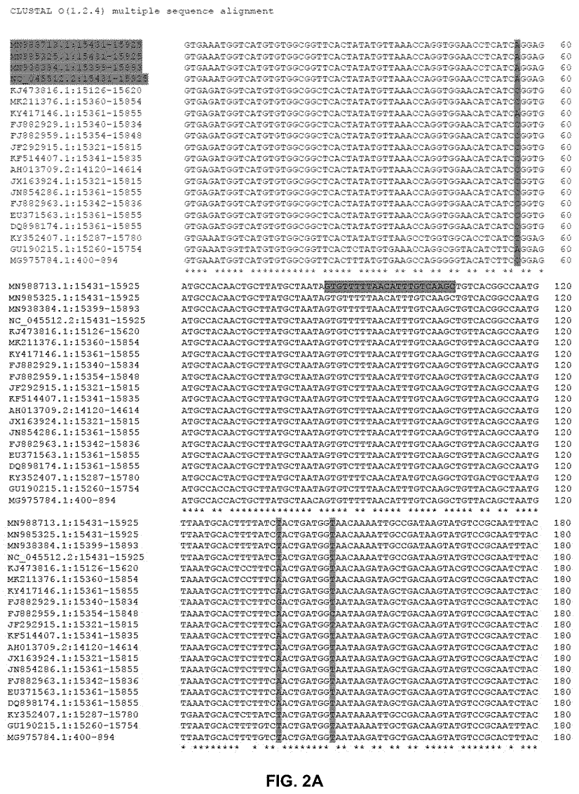

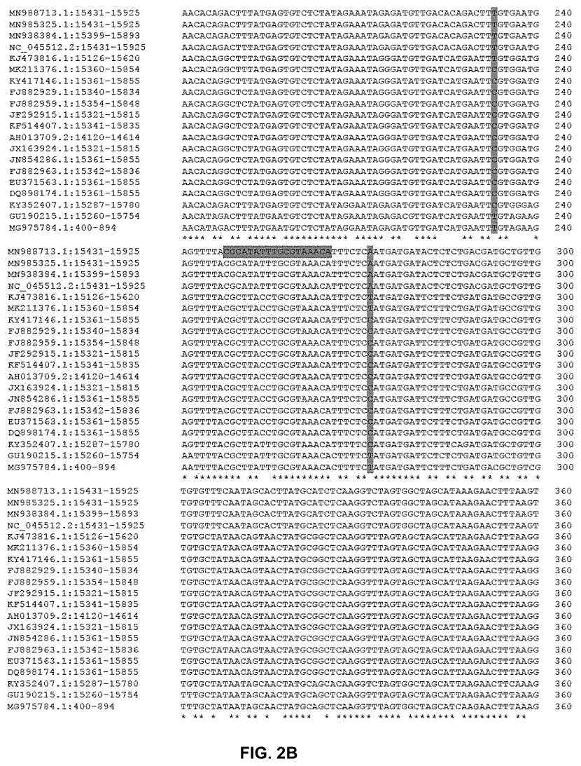

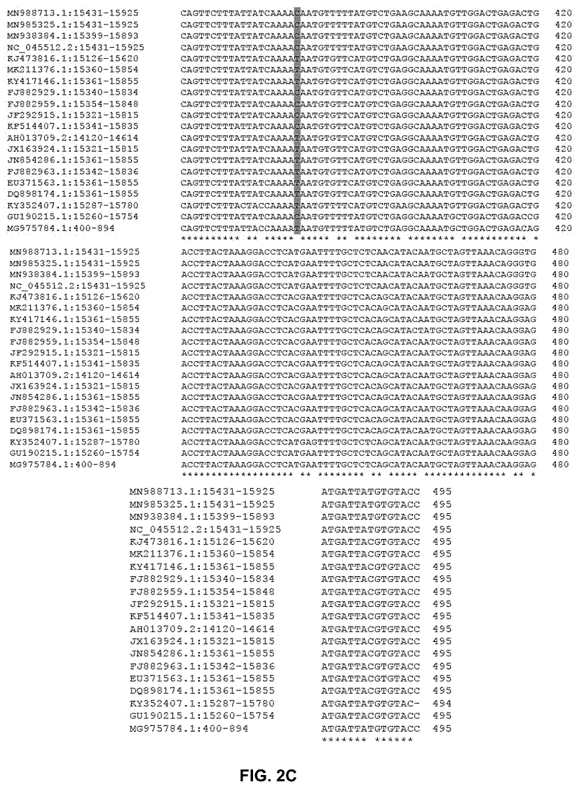

SEQ ID NO:1 conserved region of RdRP gene of MN988713.1:15431-15925 Wuhan seafood market pneumonia virus isolate 2019-nCoV/USA-IL1/2020 in FIGS. 2A-2C
SEQ ID NO:2 conserved region of RdRP gene of MN985325.1:15431-15925 Wuhan seafood market pneumonia virus isolate 2019-nCoV/USA-WA1/2020 in FIGS. 2A-2C
SEQ ID NO:3 conserved region of RdRP gene of MN938384.1:15399-15893 Wuhan seafood market pneumonia virus isolate 2019-nCoV_HKU-SZ-002a_2020 in FIGS. 2A-2C
SEQ ID NO:4 conserved region of RdRP gene of NC_045512.2:15431-15925 Wuhan seafood market pneumonia virus isolate Wuhan-Hu-1 in FIGS. 2A-2C
SEQ ID NO:5 conserved region of RdRP gene of KJ473816.1:15126-15620 BtRs-BetaCoV/YN2013 in FIGS. 2A-2C
SEQ ID NO:6 conserved region of RdRP gene of MK211376.1:15360-15854 Coronavirus BtRs-BetaCoV/YN2018B in FIGS. 2A-2C
SEQ ID NO:7 conserved region of RdRP gene of KY417146.1:15361-15855 Bat SARS-like coronavirus isolate Rs4231 in FIGS. 2A-2C
SEQ ID NO:8 conserved region of RdRP gene of FJ882929.1:15340-15834 SARS coronavirus ExoN1 isolate P3pp1 in FIGS. 2A-2C
SEQ ID NO:9 conserved region of RdRP gene of FJ882959.1:15354-15848 SARS coronavirus MA15 ExoN1 isolate P3pp6 in FIGS. 2A-2C
SEQ ID NO:10 conserved region of RdRP gene of JF292915.1:15321-15815 SARS coronavirus MA15 isolate d4ym5 in FIGS. 2A-2C
SEQ ID NO:11 conserved region of RdRP gene of KF514407.1:15341-15835 SARS coronavirus ExoN1 strain SARS/VeroE6_lab/USA/ExoN1_c5.7P20/2010 in FIGS. 2A-2C
SEQ ID NO:12 conserved region of RdRP gene of AH013709.2:14120-14614 SARS coronavirus Sin_WNV in FIGS. 2A-2C
SEQ ID NO:13 conserved region of RdRP gene of JX163924.1:15321-15815 SARS coronavirus isolate Tor2/FP1-10851 in FIGS. 2A-2C
SEQ ID NO:14 conserved region of RdRP gene of JN854286.1:15361-15855 SARS coronavirus HKU-39849 isolate recSARS-CoV HKU-39849 in FIGS. 2A-2C
SEQ ID NO:15 conserved region of RdRP gene of FJ882963.1:15342-15836 SARS coronavirus P2 in FIGS. 2A-2C
SEQ ID NO:16 conserved region of RdRP gene of EU371563.1:15361-15855 SARS coronavirus BJ182-8 in FIGS. 2A-2C
SEQ ID NO:17 conserved region of RdRP gene of DQ898174.1:15361-15855 SARS coronavirus strain CV7 in FIGS. 2A-2C
SEQ ID NO:18 conserved region of RdRP gene of KY352407.1:15287-15780 Severe acute respiratory syndrome-related coronavirus strain BtKY72 in FIGS. 2A-2C
SEQ ID NO:19 conserved region of RdRP gene of GU190215.1:15260-15754 Bat coronavirus BM48-31/BGR/2008 in FIGS. 2A-2C
SEQ ID NO:20 conserved region of RdRP gene of MG975784.1:400-894 Coronavirus SarBatCoV1 RNA-dependent RNA polymerase (RdRp) mRNA in FIGS. 2A-2C
SEQ ID NO:21 Whole genome of Wuhan SARS-CoV-2 strain in FIGS. 1A-1F
SEQ ID NO:22 Forward internal control primer (IC_RPP30-F) in Table 1
SEQ ID NO:23 Reverse internal control primer (IC_RPP30-R) in Table 1
SEQ ID NO:24 Forward SARS-CoV-2 primer (WUFor) in Table 1
SEQ ID NO:25 Reverse SARS-CoV-2 primer (WURev) in Table 1
SEQ ID NO:26 Forward primer 2 in Table 5
SEQ ID NO:27 Reverse primer 2 in Table 5
SEQ ID NO:28 Reverse primer 3 in Table 5

### DETAILED DESCRIPTION

The present invention is predicated on the surprising discovery of SNPs within a conserved region of the viral RdRP gene which facilitate the rapid and cost effective detection of severe acute respiratory syndrome coronavirus 2 (SARS-CoV-2) in samples, such as biological samples obtained from human subjects.

In a broad form, the method includes the step of analysing a conserved region of a RNA-dependent RNA polymerase (RdRP) gene or gene product of SARS-CoV-2 from a sample for the presence or absence of at least one single nucleotide polymorphism (SNP) therein.

Accordingly, in one aspect, the invention provides a method for detecting SARS-CoV-2 in a sample, said method including the step of analysing at least a portion of a viral RdRP gene or gene product from the sample for the presence or absence of at least one SNP,

wherein the at least one SNP in the at least a portion of the viral RdRP gene or gene product is at a position corresponding to at least one of positions 56, 137, 146, 233, 272 and 380 of the nucleic acid sequence set forth in any one of SEQ ID NOs: 1-4;

wherein SARS-CoV-2 is detected in the sample based on the presence or absence of the at least one SNP.

The terms “severe acute respiratory syndrome coronavirus 2” and “SARS-CoV-2” refer to the coronavirus that is widely recognized as the etiologic agent of the COVID-19 pandemic that was first identified in Wuhan of the Hubei province of China. It is envisaged that the term encompasses all isolates and strains of SARS-CoV-2 as are known in the art. An exemplary genome of a SARS-CoV-2 strain is provided in SEQ ID NO:21 in FIGS. 1A-1F.

The term “RNA-dependent RNA polymerase” as used throughout the present application relates to the RNA-dependent RNA polymerase needed for RNA replication and viral RNA synthesis, such as the single stranded RNA version of RdRP of SARS-CoV-2.

By “gene” is meant a unit of inheritance that occupies a specific locus on a genome and consists of transcriptional and/or translational regulatory sequences and/or a coding region and/or non-translated sequences (i.e., introns, 5′ and 3′ untranslated sequences). By “gene product” is meant a product of the recited gene. By way of example, gene products can include protein and cDNA sequences derived from viral RNA gene sequences, such as the RdRP protein or cDNA.

As used herein, the term “single nucleotide polymorphism” (SNP) refers to nucleotide sequence variations that occur when a single nucleotide (A, T, C or G) in the genome sequence is altered (such as via substitutions, addition or deletion). SNPs can occur in both coding (gene) and noncoding regions of the genome such as the genome of a prokaryotic or eukaryotic microorganism.

As used herein, “corresponding” nucleic acid positions or nucleotides refer to positions or nucleotides that occur at aligned loci of two or more nucleic acid molecules. Related or variant polynucleotides can be aligned by any method known to those of skill in the art. Such methods typically maximise matches, and include methods such as using manual alignments and by using the numerous alignment programs available (e.g., BLASTN) and others known to those of skill in the art. By aligning the sequences of polynucleotides, one skilled in the art can identify corresponding nucleotides or positions using identical nucleotides as guides. For example, by aligning sequences of the gene encoding the conserved region of the RdRP gene (set forth in SEQ ID NOs:1-4) with the RdRP gene from another coronavirus strain, one of skill in the art can identify corresponding positions and nucleotides using conserved nucleotides as guides.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which identical nucleic acid base (e.g., A, T, C, G) occurs in both sequence to yield the number of matched positions, dividing the number of matched positions by the total number of positions in the window of comparison (i.e., the window size), and multiplying the result by 100 to yield the percentage of sequence identity (% seq. identity).

“Homology” refers to the percentage number of nucleic acids or amino acids that are identical or constitute conservative substitutions. Homology can be determined using sequence comparison programs such as GAP (Deveraux et al., 1984, Nucleic Acids Research 12, 387-395), which is incorporated herein by reference. In this way, sequences of a similar or substantially different length to those cited herein could be compared by insertion of gaps into the alignment, such gaps being determined, for example, by the comparison algorithm used by GAP.

The term “nucleic acid” as used herein designates single- or double-stranded mRNA, RNA, cRNA, RNAi, siRNA and DNA inclusive of cDNA, mitochondrial DNA (mtDNA) and genomic DNA. The present invention also contemplates nucleic acids that have been modified such as by taking advantage of codon sequence redundancy. In a more particular example, codon usage may be modified to optimize expression of a nucleic acid in a particular organism or cell type. The invention further provides use of modified purines (for example, inosine, methylinosine and methyladenosine) and modified pyrimidines (for example, thiouridine and methylcytosine) in isolated nucleic acids of the invention.

The nucleotide symbols are set forth in the following table:

Nucleotide Symbols

| Symbol | Description |
|---|---|
| A | Adenosine |
| C | Cytidine |
| G | Guanosine |
| T | Thymidine |
| U | Uridine |
| M | Amino (adenosine, cytosine) |
| K | Keto (guanosine, thymidine) |
| R | Purine (adenosine, guanosine) |
| Y | Pyrimidine (cytosine, thymidine) |
| N | Any nucleotide |

As can be observed from FIGS. 2A-2C, SARS-CoV-2 can be identified or detected in a sample by analysing nucleic acid from the sample for one or more SNPs in the RdRP gene or a cDNA copy thereof at positions corresponding to positions 56, 137, 146, 233, 272 and 380 of the RdRP gene set forth in any one of SEQ ID NOs:1-4, wherein: an A at position 56, a T at position 137, a T at position 146, a T at position 233, an A at position 272 and/or a C at position 380 indicate that the virus is SARS-CoV-2. At least one said SNP, at least two said SNPs, at least three said SNPs, at least four said SNPs, at least five said SNPs, or at least six said SNPs, may be used in the methods of the present invention.

In particular embodiments and based on the primer sequences in SEQ ID NOs: 24 and 25, the at least one SNP in the at least a portion of the viral RdRP gene or gene product is at a position corresponding to at least one of positions 137, 146 and 233 of the nucleic acid sequence set forth in any one of SEQ ID NOs: 1-4 (i.e., a T at position 137, a T at position 146, and/or a T at position 233). At least one said SNP, at least two said SNPs, or at least three said SNPs may be used for the methods described herein.

In other embodiments and based on the primer sequences in SEQ ID NOs: 26 and 25, the at least one SNP in the at least a portion of the viral RdRP gene or gene product is at a position corresponding to at least one of positions 56, 137, 146 and 233 of the nucleic acid sequence set forth in any one of SEQ ID NOs: 1-4 (i.e., an A at position 56, a T at position 137, a T at position 146, and/or a T at position 233). At least one said SNP, at least two said SNPs, at least three said SNPs or at least four said SNPs may be used for the methods described herein.

In various embodiments and based on the primer sequences in SEQ ID NOs: 24 and 27, the at least one SNP in the at least a portion of the viral RdRP gene or gene product is at a position corresponding to at least one of positions 137, 146, 233 and 272 of the nucleic acid sequence set forth in any one of SEQ ID NOs: 1-4 (i.e., a T at position 137, a T at position 146, a T at position 233 and/or an A at position 272). At least one said SNP, at least two said SNPs, at least three said SNPs or at least four said SNPs may be used for the methods described herein.

In some embodiments and based on the primer sequences in SEQ ID NOs: 24 and 28, the at least one SNP in the at least a portion of the viral RdRP gene or gene product is at a position corresponding to at least one of positions 137, 146, 233, 272 and 380 of the nucleic acid sequence set forth in any one of SEQ ID NOs: 1-4 (i.e., a T at position 137, a T at position 146, a T at position 233, an A at position 272 and/or a C at position 380). At least one said SNP, at least two said SNPs, at least three said SNPs, at least four said SNPs or at least five said SNPs may be used for the methods described herein.

In certain embodiments and based on the primer sequences in SEQ ID NOs: 26 and 28, the at least one SNP in the at least a portion of the viral RdRP gene or gene product is at a position corresponding to at least one of positions 56, 137, 146, 233, 272 and 380 of the nucleic acid sequence set forth in any one of SEQ ID NOs: 1-4 (i.e., an A at position 56, a T at position 137, a T at position 146, a T at position 233, an A at position 272 and/or a C at position 380). At least one said SNP, at least two said SNPs, at least three said SNPs, at least four said SNPs, at least five said SNPs or at least six said SNPs may be used for the methods described herein.

It is envisaged that the particular steps and/or techniques of isolating a sample, such as a biological sample from a subject, processing a sample, genomic DNA extraction, RNA extraction, DNA detection and characterisation, RNA detection and characterisation, DNA sequencing, DNA sequence analyses, SNP genotyping studies, RNA location and identification, RNA profiling and RNA screening, RNA sequencing, RNA sequence analyses can be carried out in any suitable way known in the art.

Any method known in the art to detect one or more SNPs can be used in the methods described herein to detect and/or identify SARS-CoV-2 within a sample. In particular embodiments, the methods also facilitate in the narrowing down or, in some cases, confirming of SARS-CoV-2 over another coronavirus strain or isolate. Numerous methods are known in the art for determining the nucleotide occurrence at a particular position corresponding to a single nucleotide polymorphism in a sample. The various tools for the detection of polymorphisms include, but are not limited to, DNA sequencing, scanning techniques, hybridization based techniques, extension based analysis, high-resolution melting analysis, incorporation based techniques, restriction enzyme based analysis and ligation based techniques.

The nucleic acid, such as a viral RdRP gene or gene product, that is analysed according to the methods of the present invention may be analysed while within the sample, or may first be extracted from the sample, e.g., isolated from the sample prior to analysis. Any method for isolating nucleic acid from a sample can be used in the methods of the present invention, and such methods are well known to those of skill in the art. The extracted nucleic acid can include DNA and/or RNA (including mRNA or rRNA).

In some instances, the methods described herein include amplification of the nucleic acid. By way of example, the step of analysing comprises amplifying a nucleic acid of the viral RdRP gene or gene product from genetic material obtained from the sample to form an amplification product. “Amplification product” or “amplicon” refers to a nucleic acid product generated by nucleic acid amplification techniques. The nucleic acid may be amplified by any method known in the art including, but not limited to polymerase chain reaction (PCR), ligase chain reaction (LCR) and reverse transcription-polymerase chain reaction (RT-PCR) using one or more oligonucleotides/primers that will amplify transcribed RNA. To this end, a further step of reverse transcription can be included in the methods prior to analysis. Thus, the nucleic acid to be analysed can include the RdRP gene or a DNA copy of the RdRP gene, a cDNA copy of the RdRP gene or any combination thereof. The nucleic acid can also contain portions of the RdRP gene, provided the portions contain the nucleic acid positions that are being analysed for SNPs.

Suitable nucleic acid amplification techniques are well known to a person or ordinary skill in the art, and include polymerase chain reaction (PCR) as for example described in Ausubel et al., Current Protocols in Molecular Biology (John Wiley & Sons, Inc. 1994-1998, strand displacement amplification (SDA) as for example described in U.S. Pat. No. 5,422,252, rolling circle replication (RCR) as for example described in in Liu et al, 1996, J. Am. Chem. Soc. 118: 1587-1594 and in WO 92/01813 and WO 97/19193, nucleic acid sequence-based amplification (NASBA) as for example described in Sooknanan et al., 1994, Biotechniques 17:1077-1080, ligase chain reaction (LCR), simple sequence repeat analysis (SSR), branched DNA amplification assay (b-DNA), transcription amplification and self-sustained sequence replication, and Q-β replicase amplification as for example described in Tyagi et al., 1996, Proc. Natl. Acad. Sci. USA 93:5395-5400.

Given the single stranded RNA (ssRNA) nature of the viral RdRP gene of SARS-CoV-2, amplifying the nucleic acid of the viral RdRP gene or gene product suitably utilizes reverse transcriptase-polymerase chain reaction (RT-PCR) to generate a corresponding cDNA template of the RdRP gene suitable for amplification by standard PCR techniques, such as those disclosed herein. To this end, the present method may further include the steps of obtaining or isolating RNA, such as total RNA, from the sample in question and reverse transcribing the isolated RNA to obtain cDNA.

Such methods can utilise one or more oligonucleotide probes or primers, including, for example, an amplification primer pair, such as those provided in SEQ ID NOs: 24 and 25, SEQ ID NOs: 26 and 25, SEQ ID NOs: 24 and 27, SEQ ID NOs: 24 and 28 or SEQ ID NOs: 26 and 28, that selectively hybridize to a target polynucleotide, which contains one or more SNPs. Oligonucleotide probes useful in practicing a method of the invention can include, for example, an oligonucleotide that is complementary to and spans a portion of the target polynucleotide, including the position of the SNP, which the presence of a specific nucleotide at the polymorphic site (i.e., the SNP) is detected by the presence or absence of selective hybridization of the probe or selective amplification of the target nucleic acid. Such a method can further include contacting the target polynucleotide and hybridized oligonucleotide with an endonuclease, and detecting the presence or absence of a cleavage product of the probe, depending on whether the nucleotide occurrence at the polymorphic site is complementary to the corresponding nucleotide of the probe.

In some embodiments of the present invention, the methods suitably utilise an amplification primer or primer pair that selectively hybridize to a target polynucleotide containing one or more of the SNPs as described herein. With respect to the methods and isolated primers described herein, one or both of a sense primer and an anti-sense primer may include or encompass one or more of the SNPs present in the conserved region of the RdRP gene set forth in any one of SEQ ID NOs:1-4, such as one or more of those SNPs at positions 56, 137, 146, 233, 272 and 380 of the nucleic acid sequence set forth in any one of SEQ ID NOs: 1-4.

In particular embodiments, a primer of the invention hybridizes to or is complementary to a portion of the conserved region of the RdRP gene or gene product that includes a SNP at position 56 of the nucleic acid sequence set forth in any one of SEQ ID NOs: 1-4.

In other embodiments, a primer of the invention hybridizes to or is complementary to a portion of the conserved region of the RdRP gene or gene product that includes a SNP at position 137 and/or 146 of the nucleic acid sequence set forth in any one of SEQ ID NOs: 1-4.

In certain embodiments, a primer of the invention hybridizes to or is complementary to a portion of the conserved region of the RdRP gene or gene product that includes a SNP at position 233 of the nucleic acid sequence set forth in any one of SEQ ID NOs: 1-4.

In some embodiments, a primer of the invention hybridizes to or is complementary to a portion of the conserved region of the RdRP gene or gene product that includes a SNP at position 272 of the nucleic acid sequence set forth in any one of SEQ ID NOs: 1-4.

In specific embodiments, a primer of the invention hybridizes to or is complementary to a portion of the conserved region of the RdRP gene or gene product that includes a SNP at position 380 of the nucleic acid sequence set forth in any one of SEQ ID NOs: 1-4.

In some embodiments, the primers of the invention hybridize to or are complementary to a portion of the conserved region of the RdRP gene or gene product that includes one or more further SNPs or polymorphic regions, such as those residue positions denoted by a “.” in FIGS. 1A-1F. By way of example, the sense primer provided in SEQ ID NO:24 includes a further SNP at position 89, 92, 98 and 104 of the nucleotide sequence in SEQ ID NOs:1-4, whilst the anti-sense primer provided in SEQ ID NO:25 includes further SNPs at positions 251, 254 and 255 of the nucleotide sequence in SEQ ID NOs:1-4. The basis for this is that primers or oligonucleotides with one or more mismatched residues will not function as primers in a PCR under appropriate conditions. As a result, if nucleic acid having a given SNP in the RdRP gene of SARS-CoV-2 is present in the sample, the primer pair specific to that SNP will produce an amplification product but not to nucleic acid from or specific to another viruses, such as other coronavirus strains or isolates, that do not have the specific SNP(s) in their RdRP gene.

Any method useful for the detection of SNPs can be used in the present invention, and many different methods are known in the art for SNP genotyping (for review see Syvänen, A. C., 2001, Nat. Rev. Genet. 2, 930-942; Kim S. & Misra A. 2007, Ann Rev Biomed Eng. 9:289-320). Such methodology may consist of the use of three steps in succession, including a “reaction” (e.g., hybridization, ligation, extension and cleavage) followed by “separation” (e.g., solid phase microtitre plates, microparticles or arrays, gel electrophoresis, solution-phase homogenous or semi-homogenous). No single ideal SNP genotyping method exists for all applications, and it is well within the skill of a skilled artisan to determine the most appropriate method given the various parameters, such as sample size and number of SNPs to be analysed.

Example technologies that particularly lend themselves to clinical use and that rely on querying small numbers of SNPs, are fast, sensitive (through amplification of nucleic acid in the sample), one-step, output measured in real-time, able to be multiplexed and automated, comparatively inexpensive, and accurate include, but are not limited to, TaqMan® assays (5′ nuclease assay, Applied Biosystems), high-resolution melt analysis, molecular beacon probes such as LUX® (Invitrogen) or Scorpion® probes (Sigma Aldrich), and Template Directed Dye Incorporation (TDI, Perkin Elmer).

For example, TaqMan® (Applied Biosystems) uses a combination of hybridization with allele-specific probes, solution phase homogenous, and fluorescence resonance energy transfer. The TaqMan® assay relies on forward and reverse primers and Taq DNA polymerase to amplify nucleic acid in conjunction with 5′-nuclease activity of Taq DNA polymerase to degrade a labelled probe designed to bind across the SNP site(s). Reaction, separation and detection can all be performed at the same time and results read in real-time as the reaction proceeds. While such an approach does not lend itself to analysing large numbers of SNPs simultaneously it is particularly suitable for querying small numbers of SNPs quickly, sensitively and accurately at a reasonable cost.

Although some methods may be more suitable than others, any method known in the art to detect one or more SNPs can be used in the methods described herein to detect and/or identify SARS-CoV-2 in a sample, inclusive of those methods described in International Application No. PCT/AU2018/050471 (published as WO2018/209398), which is incorporated by reference in its entirety herein. Non-limiting examples of such methods include high resolution melt analysis, 5′ nuclease digestion, molecular beacons, oligonucleotide ligation, microarray, restriction fragment length polymorphism, antibody detection methods, direct sequencing or any combination thereof.

In particular embodiments, the methods of the present invention utilise high-resolution melting (HRM) analysis for detecting and/or identifying SARS-CoV-2 in a sample based on the SNP(s) described herein within the RdRP gene or within a DNA/cDNA copy thereof.

HRM is based upon the accurate monitoring of changes in fluorescence as a PCR product (i.e., amplicon) stained with an intercalating fluorescent dye is heated through its melting temperature (Tm). In contrast to traditional melting, the information in HRM analysis is contained in the shape of the melting curve, rather than just the calculated Tm, so HRM may be considered a form of spectroscopy. HRM analysis is a single step and closed tube method, the amplification and melting can be run as a single protocol on a real-time PCR machine.

In embodiments of the present invention, the methods utilise an amplification primer pair that selectively hybridize to a target polynucleotide containing one or more of the SNPs as described herein. The amplification reaction mixture contains the fluorescent dye, which is incorporated into the resulting amplicon. The resulting amplicon is then subjected to HRM with incremental increases in temperature (i.e., 0.01-0.5° C.) ranging from about 50° C. to about 20 95° C. At some point during this process, the melting temperature of the amplicon is reached and the two strands of DNA separate or “melt” apart.

The HRM is monitored in real-time using the fluorescent dye incorporated into the amplicon. The level of fluorescence of the dye is monitored as the temperature increases with the fluorescence reducing as the amount of double stranded DNA reduces. Changes in fluorescence and temperature can be plotted in a graph known as a melt curve.

As a skilled addressee will understand, the Tm of the amplicon at which the two DNA strands separate is predictable, being dependent on the sequence of the nucleotide bases forming the amplicon. Accordingly, it is possible to differentiate between amplicons including an amplicon containing a polymorphism (i.e., a SNP or SNPs) as the melt curves will appear different. Indeed, in some embodiments, it is possible to differentiate between amplicons containing the same polymorphism based on differences in the surrounding DNA sequences.

HRM curves can be discriminated from one another by many different strategies. For example, in many cases, HRM curves can be discriminated on the basis of obvious differences in curve shape and/or on the basis of Tm with a difference of 0.2° C. being regarded as significant. In other cases, a difference graph analysis can be used in which a defined curve is used as a baseline with other normalised curves being plotted in relation to the baseline (see Price, E. P., et al., 2007, Appln. Environ. Microbiol, 72:7793-7803). In yet other cases, a difference graph-based method can be used involving deriving the 3rd and 97th centiles from the mean±1.96 standard deviations for the fluorescence at every temperature (see Andersson, P., et al., 2009, Antimicrob. Agents Chemother. 53:2679-2683 and Merchant-Patel, S., et al., 2008, Int. J. Food Microbiol., 128:304-308).

For the methods of the invention described herein, the mere presence or absence of a suitable melt curve, such as shown in FIG. 6, after initial amplification steps with SARS-CoV-2 specific primers can be sufficient to detect SARS-CoV-2 within the sample (e.g., detect the presence of the target nucleic acid molecule amplified from the RdRP gene or gene product of SARS-CoV-2. In particular embodiments, however, the melt curve produced from the sample may be compared to a melt curve produced from a positive control nucleic acid (e.g., RNA or cDNA isolated from a SARS-CoV-2 strain or isolate).

By “primer” it is meant an oligonucleotide which, when paired with a strand of DNA, is capable of initiating the synthesis of a primer extension product in the presence of a suitable polymerizing agent, such as a DNA polymerase (e.g., Taq polymerase, RNA-dependent DNA polymerase, Sequenase™). The primer is suitably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 10 to 35 or more nucleotide residues (e.g., about 10, 11, 12, 13, 14, 15, 16, 17, 18, 19, 20, 21, 22, 23, 24, 25, 26, 27, 28, 29, 30, 31, 32, 33, 34, 35 nucleotides in length including any range therein), although it can contain fewer nucleotide residues. Primers can be large polynucleotides, such as from about 200 nucleotides to several kilobases or more. Suitably, the GC ratio of a primer should be above 30%, 35%, 40%, 45%, 50%, 55%, or 60% so as to prevent hair-pin structures forming on the primer. Furthermore, the amplicon or nucleic acid to be amplified should be sufficiently long enough to be detected by standard molecular biology methodologies. Suitably, the amplicon or amplified nucleic acid is at least about 20, 25, 30, 35, 40, 45, 50, 55, 60, 65, 70, 75, 80, 85, 90, 95, 100, 110, 120, 130, 140, 150, 175, 200, 250, 300, 350, 400, 450, 500, 550, 600, 700, 800, or 1000 base pairs in length.

Primers can be selected to be “substantially complementary” to the sequence on the template to which it is designed to hybridize and serve as a site for the initiation of synthesis. By “substantially complementary” it is meant that the primer is sufficiently complementary to hybridize with a target polynucleotide. In some embodiments, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

Primers may be manufactured using any convenient method of synthesis. Examples of such methods may be found in “Protocols for Oligonucleotides and Analogues; Synthesis and Properties”, Methods in Molecular Biology Series, Volume 20, Ed. Sudhir Agrawal, Humana ISBN: 0-89603-247-7, 1993. The primers may also be labelled to facilitate detection.

Exemplary primer sequences are provided in SEQ ID NOs:22 to 28 shown in Tables 1 and 5.

In various embodiments, the step of amplifying the nucleic acid of the viral RdRP gene or gene product includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:25, a nucleotide sequence complementary thereto or a fragment or variant thereof.

In other embodiments, the step of amplifying the nucleic acid of the viral RdRP gene or gene product includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:25, a nucleotide sequence complementary thereto or a fragment or variant thereof.

In various embodiments, the step of amplifying the nucleic acid of the viral RdRP gene or gene product includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:27, a nucleotide sequence complementary thereto or a fragment or variant thereof.

In some embodiments, the step of amplifying the nucleic acid of the viral RdRP gene or gene product includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof.

In particular embodiments, the step of amplifying the nucleic acid of the viral RdRP gene or gene product includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof.

As used herein, “variant” nucleic acids refer to nucleic acids that comprise nucleotide sequences of naturally occurring (e.g., allelic) variants and orthologs (e.g., from a different strain or isolate) of SARS-CoV-2. Preferably, nucleic acid variants share at least 70% or 75%, preferably at least 80% or 85% or more preferably at least 90%, 91%, 92%, 93%, 94%, 95%, 96%, 97%, 98% or 99% sequence identity with a nucleotide sequence disclosed herein.

Also included are nucleic acid fragments. A “fragment” is a segment, domain, portion or region of a nucleic acid, which respectively constitutes less than 100% of the nucleotide sequence. In particular embodiments, a nucleic acid fragment may comprise, for example, at least 5, 6, 7, 8, 9, 10, 11, 12, 13, 14, 15, 16, 17, 18, 19, 20, 21, 25, 3035, 40, 45, 50, 55, 60, 65, 70, 75, 80, 85, 90, 95, 100, 125, 150, 175, 200, 225, 250, 275, 300, 325, 350, 375, 400, 425, 450, 475, 500, 600, 700 and 800, 900, 1000, 1500 and 2000 contiguous nucleotides of said nucleic acid.

In particular embodiments, the methods described herein utilise a probe, such as to detect the presence or absence of one or more SNPs or an amplified nucleic acid molecule. “Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labelled directly or indirectly.

In alternative embodiments, however, the methods of the invention can be performed without the use of a probe, inclusive of labelled probes, such as in those embodiments that utilise HRM.

It will be well appreciated by a person of skill in the art that the isolated nucleic acids, such as isolated oligonucleotides and primers, of the invention can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008).

In some embodiments, complementary nucleic acids hybridise to nucleic acids of the invention under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Stringent conditions are well-known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

The primers and probes of the present invention suitably hybridize to at least a portion of the RdRP gene of SARS-CoV-2 (or DNA/cDNA copies thereof) containing the SNP position(s) described herein. For example, the primers may hybridize to a sequence flanking or including one or more SNPs described herein, and the probe may hybridize to a sequence that includes one or more SNPs described herein.

The sample may be or comprise an environmental sample, such as an air, soil or water sample, a filtrate, a food or manufactured product, or swap from a surface, such as from a medical instrument or workplace surface. In other examples, the sample is obtained from cultured cells.

In various embodiments, the sample referred to herein is a biological sample obtained from a subject. The term “biological sample” as used herein refers to a sample that may be extracted, untreated, treated, diluted or concentrated from a patient or subject. Suitably, the biological sample is selected from any part of a patient or subject's body, including, but not limited to, cells, hair, skin, nails, tissues or bodily fluids, secretions or excretions, such as sputum, saliva, cerebrospinal fluid, serum, plasma, stool, urine and blood. In particular embodiments, the sample is or comprises a nasopharyngeal aspirate or swab.

In further embodiments, the methods described herein further include the initial step of obtaining the sample, such as obtaining the biological sample from the subject in question.

Accordingly, the aforementioned methods may be used for identifying or diagnosing subjects or patients currently or previously infected with SARS-CoV-2. To this end, the methods of the present invention are particularly useful in assisting clinicians in determining whether the subject has a SARS-CoV-2 infection and if so, an appropriate course of action or treatment based on this diagnosis.

As used herein, the terms “subject” and “patient” are used interchangeably. However, it will be understood that “patient” does not imply that symptoms are present. As used herein, the terms “subject” and “subjects” refer to an animal, preferably a mammal including a non-primate (e.g., cows, pigs, horses, goats, sheep, cats, dogs, avian species and rodents) and a primate (e.g., monkeys such as a cynomolgous monkey and humans), and more particularly a human.

In a related aspect, the invention resides in a method for detecting severe acute respiratory syndrome coronavirus 2 (SARS-CoV-2) in a sample, said method including the steps of:
(a) amplifying a nucleic acid from a conserved region of a RNA-dependent RNA polymerase (RdRP) gene or gene product of SARS-CoV-2; and
(b) detecting the presence or absence of the nucleic acid amplified in step (a).

The terms “conserved region” or “conserved sequence” as used herein refer to a segment of nucleotide sequence of a gene or amino acid sequence of a protein that is significantly similar between various different nucleotide sequences of the particular gene or protein, such as that of different coronavirus strains or isolates. It is envisaged that the conserved region of the RdRP gene or gene product of SARS-CoV-2 can be determined by any means or method known in the art. In particular embodiments, however, the conserved region of the RdRP gene or gene product contains one or more SNPs that are specific to of SARS-CoV-2, such as that conserved region set forth in the SEQ ID NOs:1-4. By way of example, the nucleic acid amplified in step (a) can include at least one SNP, wherein the at least one SNP is at a position corresponding to at least one of positions 56, 137, 146, 233, 272 and 380 of the nucleic acid sequence set forth in any one of SEQ ID NOs: 1-4. In some embodiments, the nucleic acid amplified in step (a) includes at least one SNP at a position corresponding to at least one of positions 137, 146 and 233 of the nucleic acid sequence set forth in any one of SEQ ID NOs: 1-4.

It will be appreciated that step (a) of the present method may be performed by any means or method known in the art, such as those described herein. In particular embodiments, however, the step of amplifying the nucleic acid of the RdRP gene or gene product at least partly comprises or utilizes reverse transcriptase PCR (RT-PCR) or an RT-PCR step to generate a cDNA template of the viral ssRNA RdRP gene suitable for amplification by standard PCR techniques, such as those provided herein. To this end, the present method may further include the steps of obtaining or isolating RNA, such as total RNA, from the sample in question and reverse transcribing the isolated RNA to obtain cDNA.

The amplification step in step (a) may further utilise any suitable primers or primer pairs, such as those described herein. Suitably, the primers or primer pairs encompass or amplify a nucleic acid sequence that includes at least one SNP of those described herein (i.e., at least one of positions 56, 137, 146, 233, 272 and 380 of the nucleic acid sequence set forth in any one of SEQ ID NOs: 1-4).

In particular embodiments, the step of amplifying the nucleic acid includes using a forward primer comprising, consisting of or consisting essentially of a nucleotide sequence set forth in SEQ ID NO:24, a nucleotide sequence complementary thereto or a fragment or variant thereof and/or a reverse primer comprising, consisting of or consisting essentially of a nucleotide sequence set forth in SEQ ID NO:25, a nucleotide sequence complementary thereto or a fragment or variant thereof.

In other embodiments, the step of amplifying the nucleic acid includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:25, a nucleotide sequence complementary thereto or a fragment or variant thereof. In these embodiments, the at least one SNP may be at a position corresponding to at least one of positions 56, 137, 146 and 233 of the nucleic acid sequence set forth in any one of SEQ ID NOs: 1-4.

In various embodiments, the step of amplifying the nucleic acid includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:27, a nucleotide sequence complementary thereto or a fragment or variant thereof. In these embodiments, the at least one SNP may be at a position corresponding to at least one of positions 137, 146, 233 and 272 of the nucleic acid sequence set forth in any one of SEQ ID NOs: 1-4.

In some embodiments, the step of amplifying the nucleic acid includes using a forward or sense primer comprising, consisting of or consisting essentially of a nucleotide sequence set forth in SEQ ID NO:24, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof. In these embodiments, the at least one SNP may be at a position corresponding to at least one of positions 137, 146, 233, 272 and 380 of the nucleic acid sequence set forth in any one of SEQ ID NOs: 1-4.

In particular embodiments, the step of amplifying the nucleic acid includes using a forward or sense primer comprising, consisting of or consisting essentially of a nucleotide sequence set forth in SEQ ID NO:26, a nucleotide sequence complementary thereto or a fragment or variant thereof and/or a reverse or antisense primer comprising, consisting of or consisting essentially of a nucleotide sequence set forth in SEQ ID NO:28, a nucleotide sequence complementary thereto or a fragment or variant thereof. In these embodiments, the at least one SNP may be at a position corresponding to at least one of positions 56, 137, 146, 233, 272 and 380 of the nucleic acid sequence set forth in any one of SEQ ID NOs: 1-4.

Generally speaking, the skilled person would appreciate that the step of detecting the presence or absence of the nucleic acid amplified in step (a) can be performed by any means or method known in the art, inclusive of those hereinbefore provided. In particular embodiments, the presence or absence of the nucleic acid is detected at least in part using high resolution melt (HRM) analysis. HRM analysis advantageously allows for the simple (e.g., no use of labelled probes), rapid (e.g., less than 45 minutes) and reliable detection of SARS-CoV-2 in samples. With the portability of some newer platforms, such as the Mic qPCR cycler from Bio Molecular Systems, PCR-based amplification and HRM analysis of a target nucleic acid, and hence SARS-CoV-2 detection, can also be performed remotely or at point of care (POC) facilities, precluding the need for transport of samples to, for example, external testing laboratories.

In various embodiments, the method of the present aspect further includes the step of analysing at least a portion of the nucleic acid amplified in step (a) for the presence or absence of at least one SNP, wherein the at least one SNP is at a position corresponding to at least one of positions 56, 137, 146, 233, 272 and 380 of the nucleic acid sequence set forth in any one of SEQ ID NOs: 1-4. More particularly, the at least one SNP is suitably at a position corresponding to at least one of positions 137, 146 and 233 of the nucleic acid sequence set forth in any one of SEQ ID NOs: 1-4.

Such analysis may be performed by any means or method known in the art, such as those disclosed herein. In other embodiments, the at least one SNP is at a position corresponding to at least one of positions 56, 137, 146 and 233 of the nucleic acid sequence set forth in any one of SEQ ID NOs: 1-4. In various embodiments, the at least one SNP is at a position corresponding to at least one of positions 137, 146, 233 and 272 of the nucleic acid sequence set forth in any one of SEQ ID NOs: 1-4. In some embodiments, the at least one SNP is at a position corresponding to at least one of positions 137, 146, 233, 272 and 380 of the nucleic acid sequence set forth in any one of SEQ ID NOs: 1-4.

Suitably, the sample is a biological sample, such as cells, blood, serum, plasma, saliva, cerebrospinal fluid, urine, stool, sputum, nasopharyngeal aspirates or swabs, obtained from a subject.

In some embodiments of the aforementioned aspects, the methods of the invention further include the step of detecting or determining replication of SARS-CoV-2 in the sample. This may be achieved by any method known in the art. By way of example, the present methods may include the further step of detecting the presence or absence of SARS-CoV-2 subgenomic RNA (sgRNA) in the sample, such as by amplifying a further nucleic acid from a portion of subgenomic RNA specific to SARS-CoV-2 and subsequently detecting the further nucleic acid. An exemplary method for detecting the replication of SARS-CoV-2 is described in Australian Provisional Patent Application No. 2020902624, which is incorporated by reference in its entirety herein.

In some embodiments, the methods described generally herein are performed, at least in part, by a processing system, such as a suitably programmed computer system. A stand-alone computer, with the microprocessor executing applications software allowing the above-described methods to be performed, may be used. Alternatively, the methods can be performed, at least in part, by one or more processing systems operating as part of a distributed architecture. For example, a processing system can be used to detect the presence of an SNP at a position by detecting the hybridization of a probe to a nucleic acid molecule. A processing system also can be used to determine whether a sample (or a subject from which the sample is obtained) is positive or negative for SARS-CoV-2 on the basis of detection of one or more SNPs. In some examples, commands inputted to the processing system by a user may assist the processing system in making these determinations.

In specific embodiments, a processing system includes at least one microprocessor, a memory, an input/output device, such as a keyboard and/or display, and an external interface, interconnected via a bus. The external interface can be utilised for connecting the processing system to peripheral devices, such as a communications network, database, or storage devices. The microprocessor can execute instructions in the form of applications software stored in the memory to allow the SNP detection and/or virus detection and/or identification process to be performed, as well as to perform any other required processes, such as communicating with the computer systems. The application software may include one or more software modules, and may be executed in a suitable execution environment, such as an operating system environment, or the like.

In yet another aspect, the invention relates to an isolated probe, tool or reagent capable of detecting SARS-CoV-2 in a sample, wherein the probe, tool or reagent is capable of binding, detecting or identifying the presence or absence of at least one SNP in at least a portion of a viral RdRP gene or gene product,

wherein the at least one SNP in the at least a portion of the viral RdRP gene or gene product is at a position corresponding to at least one of positions 56, 137, 146, 233, 272 and 380 of the nucleic acid sequence set forth in any one of SEQ ID NOs: 1-4.

As used herein, by “isolated” is meant material, such as a probe, tool, reagent, oligonucleotide or primer, that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in recombinant, chemical synthetic, enriched, purified or partially purified form.

The probe, tool or reagent may be, but is not limited to, an oligonucleotide, a primer, a nucleic acid, a polynucleotide, DNA, cDNA, RNA, a peptide or a polypeptide. These may be, for example, single stranded or double stranded, naturally occurring, isolated, purified, chemically modified, recombinant or synthetic.

The probe, tool or reagent may be, but is not limited to, an antibody or other type of molecule or chemical entity capable of specifically binding, detecting or identifying at least a portion of a RdRP gene or gene product in a sample containing at least one SNP provided herein.

The probe, tool or reagent may be any number or combination of the above, and the number and combination will depend on a desired result to be achieved—e.g., detection of SNP at a genomic level (genotyping) or at the RNA transcription level.

The probe, tool or reagent may be detectably labelled. A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labelled, so as to incorporate the label into the amplification product.

In certain embodiments, the at least one probe, tool or reagent is for specifically binding, detecting or identifying at least a portion of a RdRP gene or gene product of SARS-CoV-2 in a sample containing at least one SNP as provided herein.

Said probe, tool or reagent may be a primer. Said probe, tool or reagent may comprise an oligonucleotide having a nucleotide sequence as set forth in at least one of SEQ ID NOs: 24 to 28. In one embodiment, the isolated probe, tool or reagent may comprise two primers, each of which hybridizes to at least a portion of a RdRP gene (or gene product) of SARS-CoV-2, containing a SNP as defined above. In another embodiment, the at least one probe, tool or reagent may comprise a probe that hybridizes to at least a portion of a RdRP gene (or gene product) of SARS-CoV-2, containing a SNP as defined above.

The present specification explains how various SNPs can be used as ‘tools’ for identifying, partially identifying or classifying a bacterium, yeast organism or filamentous fungi, or diagnosing a bacterial, yeast organism or filamentous fungi infection. This SNP finding enables the inventors to develop gene/allele-based and gene product-based probes, tools, reagents, methods and assays for detecting and/or identifying SARS-CoV-2 in a sample.

One of skill in the art could readily design, produce or manufacture a wide range of gene/allele-based and gene product-based probes, tools, reagents, methods and assays based on the information provided herein and especially in FIGS. 1A-1F and 2A-2C.

Generally speaking, such probes, tools or reagents based on or developed in view of the SNPs outlined in the present specification may, for example, specifically bind, detect, identify, characterise or quantify the gene or part of the gene, or other gene products or parts thereof.

Generally speaking, such probe, tool or reagent can be for detection of a polymorphism for example at the genomic level, or at the transcription level.

Generally speaking, such probe, tool or reagent can also be an antibody or other type of molecule or chemical entity capable of detecting the gene or gene product (such as RNA).

More specifically, probes, tools and reagents may include, but are not limited to, the following:
1. An isolated, purified, synthetic or recombinant form of a conserved region of the RdRP gene (e.g., ssRNA) of SARS-CoV-2, or a fragment thereof, including a fragment containing a SNP of interest—single stranded or double stranded.
2. A non-naturally occurring polynucleotide, recombinant polynucleotide, oligonucleotide or cDNA form of the RdRP gene of SARS-CoV-2, or a fragment thereof, including a fragment containing a SNP of interest—single stranded or double stranded.
3. An expression vector, recombinant cell or biological sample comprising the nucleic acid or polynucleotide of 1 or 2 above.

The probe, tool or reagent can be, but is not limited to, an antibody or other type of molecule or chemical entity capable of detecting the gene or gene product (RNA or polypeptide).

The at least one probe, tool or reagent can be any number or combination of the above, and the number and combination will depend on the desired result to be achieved—e.g. detection of a polymorphism at the genomic level (genotyping) or at the RNA level.

Suitably, the probe, tool or reagent is or comprises an oligonucleotide or primer comprising, consisting of or consisting essentially of a nucleic acid sequence as set forth in at least one of SEQ ID NOs: 24 to 28, a nucleic acid sequence complementary thereto or a fragment or variant thereof.

In one embodiment, the isolated probe, tool or reagent comprises or consists of a nucleotide sequence having at least 90%, at least 91%, at least 92%, at least 93%, at least 94%, at least 95%, at least 96%, at least 97%, at least 98%, at least 99% or 100% sequence homology or identity with the sequence as set forth in at least one of SEQ ID NOs: 24 to 28.

In still a further aspect, the invention provides an isolated oligonucleotide, primer or nucleic acid molecule comprising, consisting of or consisting essentially of a nucleic acid sequence as set forth in any one of SEQ ID NOs:24 to 28, a nucleic acid sequence complementary thereto, or a fragment or variant thereof.

The term “oligonucleotide” as used herein refers to a polymer composed of a multiplicity of nucleotide residues (deoxynucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length generally from about 10 to 35 nucleotide residues (e.g., about 10, 11, 12, 13, 14, 15, 16, 17, 18, 19, 20, 21, 22, 23, 24, 25, 26, 27, 28, 29, 30, 31, 32, 33, 34 and 35 nucleotides in length including any range therein), but the term can refer to molecules of any length, although the term “polynucleotide” or “nucleic acid” is typically used for large oligonucleotides.

In one embodiment, the isolated oligonucleotide comprises or consists of a nucleotide sequence having at least 90%, at least 91%, at least 92%, at least 93%, at least 94%, at least 95%, at least 96%, at least 97%, at least 98%, at least 99% or 100% sequence homology or identity with the sequence as set forth in at least one of SEQ ID NOs: 24 to 28.

In yet another aspect, the invention relates to an array comprising: the isolated probe, tool or reagent described herein; and/or the isolated oligonucleotide described herein.

In a related aspect, the invention provides a biochip comprising a solid substrate and the isolated probe, tool or reagent described herein; and/or the isolated oligonucleotide described herein.

In this regard, the present invention contemplates the use of an array of oligonucleotides, wherein discrete positions on the array are complementary to one or more of the sequences containing the SNPs of the present invention, e.g. oligonucleotides of at least 12 nt, at least about 15 nt, at least about 18 nt, at least about 20 nt, or at least about 25 nt, or longer, and including the sequence flanking the polymorphic position. Such an array may comprise a series of oligonucleotides, each of which c an specifically hybridize to a different polymorphism. For examples of arrays, see Hacia et al., 1996, Nat. Genet. 14: 441-447 and De Risi et al, 1996, Nat. Genet. 14: 457-460.

A nucleotide array can include all or a subset of the SNPs of the invention, as required. One or more polymorphic forms may be present in the array. The oligonucleotide sequence on the array is generally at least about 12 nt in length, at least about 15 nt, at least about 18 nt, at least about 20 nt, or at least about 25 nt, or more, such as 100 to 200 nt in length. For examples of arrays, see Ramsay, 1998, Nature Biotech. 16: 40-44; Lockhart et al., 1996, Nature Biotechnol. 14:1675-1680; Hacia et al., 1996, Nat. Genet. 14: 441-447 and De Risi et al, 1996, Nat. Genet. 14: 457-460.

A number of methods are available for creating micro-arrays of biological samples, such as arrays of DNA samples to be used in DNA hybridization assays. Examples of such arrays are discussed in detail in PCT Application No. WO95/35505; U.S. U.S. Pat. No. 5,445,934; and Drmanac et al., 1993, Science 260: 1649-1652. Yershov et al., 1996, Genetics 93: 4913-4918 describes an alternative construction of an oligonucleotide array. The construction and use of oligonucleotide arrays are reviewed by Ramsay (Ramsay, 1998, Nature Biotech. 16: 40-44).

Methods of using high-density oligonucleotide arrays for identifying polymorphisms within nucleotide sequences are known in the art. For example, Milosavljevic et al., 1996 describe DNA sequence recognition by hybridization to short oligomers (see also, Drmanac et al., 1998, Nature Biotech. 16: 54-58 and Drmanac and Drmanac, 1999, Methods Enzymol. 303:30 165-178). The use of arrays for identification of unknown mutations is proposed by Ginot 1997 Ginot (Ginot, 1997, Human Mutation 10:1-10).

Detection of known mutations is described in Hacia et al., 1996, Nat. Genet. 14: 441-447; Cronin, et al., 1996, Human Mut. 7: 244-255; and others. The use of arrays in genetic mapping is discussed in Chee, et al., 1996, Science 274: 610-613; Sapolsky and Lishutz, 1996, Genomics 33: 445-456; and Shoemaker et al., 1996, Nat. Genet. 14: 450-456.

Quantitative monitoring of gene expression patterns with a complementary DNA microarray is described in Schena et al., 1995, Science 270: 467; and DeRisi et al., 1997, Science 270:680-686. Wodicka et al., 1997 (Wodicka et al, 1997, Nat. Biotech. 15: 1-15) performs genome wide expression monitoring in S. cerevisiae.

High-density microarrays of oligonucleotides are known in the art and are commercially available. The sequence of oligonucleotides on the array will correspond to a known target sequence. The length of oligonucleotide present on the array is an important factor in how sensitive hybridization will be to the presence of a mismatch. Usually oligonucleotides will be at least about 12 nt in length, more usually at least about 15 nt in length, preferably at least about 20 nt in length and more preferably at least about 25 nt in length, and will be not longer than about 35 nt in length, usually not more than about 30 nt in length.

Methods of producing large arrays of oligonucleotides are described in U.S. Pat. Nos. 5,134,854 and 5,445,934 using light-directed synthesis techniques. Using a computer-controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in International Publication WO 95/35505.

Microarrays can be scanned to detect hybridization of the labelled genome samples. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that may be used in the subject methods is described in U.S. Pat. No. 5,631,734. A scanning laser microscope is described in Shalon, et al., 1996, Genome Res. 6: 639. A scan, using the appropriate excitation line, is performed for each fluorophore used. The digital images generated from the scan are then combined for subsequent analysis. For any particular array element, the ratio of the fluorescent signal from one nucleic acid sample is compared to the fluorescent signal from the other nucleic acid sample, and the relative signal intensity determined.

Methods for analysing the data collected by fluorescence detection are known in the art. Data analysis includes the steps of determining fluorescent intensity as a function of substrate position from the data collected, removing outliers, i.e., data deviating from a predetermined statistical distribution, and calculating the relative binding affinity of the targets from the remaining data. The resulting data may be displayed as an image with the intensity in each region varying according to the binding affinity between targets and probes.

Nucleic acid analysis via microchip technology is also applicable to the present invention. In this technique, thousands of distinct oligonucleotide probes can be applied in an array on a silicon chip. A nucleic acid to be analysed is fluorescently labelled and hybridized to the probes on the chip. It is also possible to study nucleic acid-protein interactions using these nucleic acid microchips. Using this technique, one can determine the presence of mutations, sequence the nucleic acid being analysed, or measure expression levels of a gene of interest. The method is one of parallel processing of many, even thousands, of probes at once and can tremendously increase the rate of analysis.

An array-based tiling strategy useful for detecting SNPs is described in EP 785280. Briefly, arrays may generally be “tiled” for a large number of specific polymorphisms. “Tiling” refers to the synthesis of a defined set of oligonucleotide probes that are made up of a sequence complementary to the target sequence of interest, as well as preselected variations of that sequence, e.g., substitution of one or more given positions with one or more members of the basis set of monomers, i.e., nucleotides. Tiling strategies are further described in PCT application No. WO 95/11995. In some embodiments, arrays are tiled for a number of specific SNPs. In particular, the array is tiled to include a number of detection blocks, each detection block being specific for a specific SNP or a set of SNPs. For example, a detection block may be tiled to include a number of probes that span the sequence segment that includes a specific SNP. To ensure probes that are complementary to each allele, the probes are synthesized in pairs differing at the SNP position. In addition to the probes differing at the SNP position, monosubstituted probes are also generally tiled within the detection block. Such methods can readily be applied to the SNP information disclosed herein.

These monosubstituted probes have bases at and up to a certain number of bases in either direction from the polymorphism, substituted with the remaining nucleotides (selected from A, T, G, C and U). Typically, the probes in a tiled detection block will include substitutions of the sequence positions up to and including those that are 5 bases away from the SNP. The monosubstituted probes provide internal controls for the tiled array, to distinguish actual hybridization from artificial cross-hybridization. Upon completion of hybridization with the target sequence and washing of the array, the array is scanned to determine the position on the array to which the target sequence hybridizes. The hybridization data from the scanned array is then analysed to identify which allele or alleles of the SNP are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length and the sequences complementary thereto, or a fragment thereof, the fragment comprising at least about 8 consecutive nucleotides, preferably 10, 15, 20, more preferably 25, 30, 40, 47, or 50 consecutive nucleotides and containing apolymorphic base, such as those described herein. In some embodiments the polymorphic base is within 5, 4, 3, 2, or 1 nucleotides from the centre of the polynucleotide, more preferably at the centre of the polynucleotide. In other embodiments, the chip may comprise an array containing any number of polynucleotides of the present invention.

An oligonucleotide may be synthesized on the surface of the substrate by using a chemical coupling procedure and an ink jet application apparatus, as described in PCT application WO95/251116. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number which lends itself to the efficient use of commercially available instrumentation.

Using such arrays, the present invention provides methods of identifying the SNPs of the present invention in a sample. Such methods comprise incubating a test sample with an array comprising one or more oligonucleotide probes corresponding to at least one SNP position of the present invention, and assaying for binding of a nucleic acid from the test sample with one or more of the oligonucleotide probes. Such assays will typically involve arrays comprising oligonucleotide probes corresponding to many SNP positions and/or allelic variants of those SNP positions, at least one of which is a SNP of the present invention.

Conditions for incubating a nucleic acid molecule with a test sample vary. Incubation conditions depend on the format employed in the assay, the detection methods employed, and the type and nature of the nucleic acid molecule used in the assay. One skilled in the art will recognize that any one of the commonly available hybridization, amplification or array assay formats can readily be adapted to employ the novel SNPs disclosed herein.

Multicomponent integrated systems may also be used to analyse SNPs. Such systems miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Integrated systems can be envisaged mainly when micro-fluidic systems are used. These systems comprise a pattern of micro-channels designed onto a glass, silicon, quartz, or plastic wafer included on a microchip. The movements of the samples are controlled by electric, electro-osmotic or hydrostatic forces applied across different areas of the microchip to create functional microscopic valves and pumps with no moving parts. Varying the voltage controls the liquid flow at intersections between the micro-machined channels and changes the liquid flow rate for pumping across different sections of the microchip.

For genotyping SNPs, the microfluidic system may integrate, for example, nucleic acid amplification, mini-sequencing primer extension, capillary electrophoresis, and a detection method such as laser induced fluorescence detection.

In a first step, the DNA samples can be amplified, preferably by PCR. Then, the amplification products are subjected to automated mini-sequencing reactions using ddNTPs (specific fluorescence for each ddNTP) and the appropriate oligonucleotide mini-sequencing primers which hybridize just upstream of the targeted polymorphic base. Once the extension at the 3′ end is completed, the primers are separated from the unincorporated fluorescent ddNTPs by capillary electrophoresis. The separation medium used in capillary electrophoresis can be, for example, polyacrylamide, polyethylene glycol or dextran. The incorporated ddNTPs in the single nucleotide primer extension products are identified by laser-induced fluorescence detection. This microchip can be used to process at least 96 to 384 samples, or more, in parallel.

In a final aspect, the invention resides in a kit or assay for detecting SARS-CoV-2 in a sample, said kit or assay comprising: the isolated probe, tool or reagent described herein; the isolated oligonucleotide described herein; the array described herein; and/or the biochip described herein.

In this regard, all the essential materials and reagents required for performing the methods described herein, such as detecting one or more SNPs in the RdRP gene of SARS-CoV-2, may be assembled together in a kit. The kits may also optionally include appropriate reagents for detection of labels, positive and negative controls, fluorescent dyes (e.g., for HRM analysis), washing solutions, blotting membranes, microtitre plates, dilution buffers and the like. For example, a nucleic acid-based detection kit for the identification of polymorphisms may include one or more of the following: (i) nucleic acid (e.g., RNA or cDNA) from a SARS-CoV-2 strain or isolate (which may be used as a positive control); and (ii) a primer and/or probe that specifically hybridizes to at least a portion of the RdRP gene or gene product containing the SNP position(s) to be analysed, and optionally one or more other markers, at or around the suspected SNP site. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. The kit can also feature various devices and reagents for performing one of the assays described herein; and/or printed instructions for using the kit to identify the presence of a SNP as defined herein.

In particular embodiments, the kit includes a pair of oligonucleotides (e.g., comprising 20 a forward/sense primer and an antisense/reverse primer), wherein at least one of the pair of oligonucleotides comprises, consists of or consists essentially of the nucleic acid sequence as set forth in any one of SEQ ID NOs: 24 to 28, a nucleic acid sequence complementary thereto or a fragment or variant thereof.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

The disclosure of every patent, patent application, accession number and publication cited herein is hereby incorporated herein by reference in its entirety.

In order that the invention may be readily understood and put into practical effect, particular preferred embodiments will now be described by way of the following non-limiting examples.

### EXAMPLES

### Example 1 Identification of SNPs

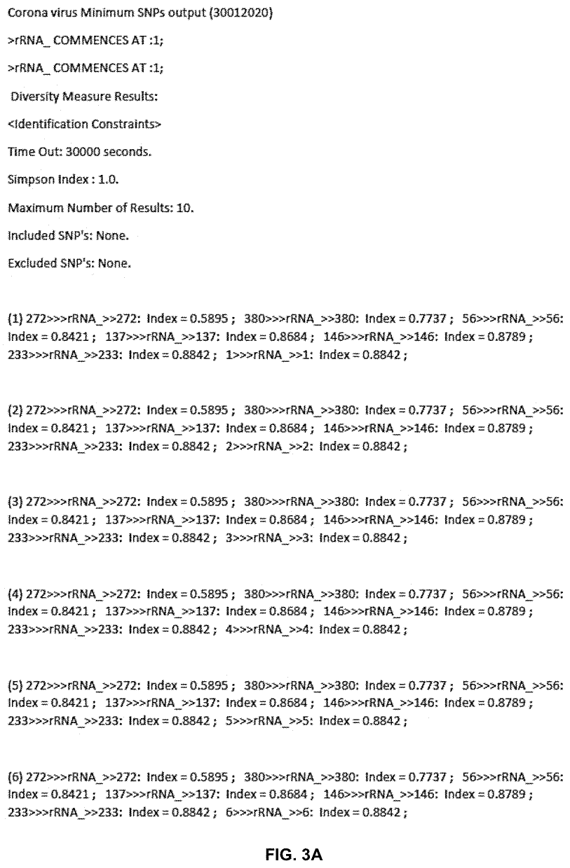

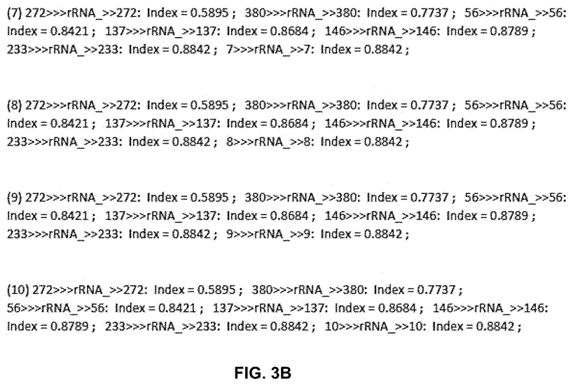

A highly conserved 495-bp region of the RNA-dependent-RNA polymerase (RdRp) gene was selected and used as input for an NCBI BLASTn analysis, which lists all SARS-Co viruses with this region. This search was limited to the first 100 SARS-Co viruses that had 100% identity match to SARS-Co viruses. From this list, ten SARS-CoV strains were randomly selected as representative of both human and animal SARS-Co viruses, and 4/10 sequences were the Wuhan seafood market pneumonia virus. Table 1 lists the ten selected strains that were used to design the synthetic RdRp SARS-CoV-2 RNA fragment used in the InfectID-COVID-19-D assay. Aligned sequences were used as input to define discriminatory SNPs (FIGS. 3A-3B).

TABLE 1
SARS-Co virus strains with associated Genbank Accession
numbers, used to design the synthetic RdRp gene region.

| | Genbank Accession |
|---|---|
| SARS-CoV strain type | number/s |
| Wuhan seafood market | |
| pneumonia virus strains: | |
| Wuhan-Hu-1 | NC_045512.2 |
| 2019-nCoV/USA- | MN988713.1 |
| IL1/2020 | MN985325.1 |
| 2019-nCoV/USA- | MN938384.1 |
| WA1/2020 | |
| HKU-SZ-002a_2020 | |
| Bat coronavirus - China (Beta- | GU190215.1 |
| CoV) BM48-31/BGR/2008 | |
| SARS corona virus - Urbani strain - | AY278741.1 |
| Asian human SARS (2003)_1 | |
| SARS corona virus - mouse adapted | FJ882959.1 |
| coronavirus (2007) | |
| SARS coronavirus strain CV7 | DQ898174.1 |
| (human clinical isolate) | |
| SARS coronavirus strain from | KY352407.1 |
| Kenyan bats, BtKY72 | |
| SARS - Bat corona virus in European | EU371563.1 |
| bats (Europe 2013): J182-8 | |

### Example 2

The present example illustrates an embodiment of a one-step PCR test that can be utilised to detect the presence or absence of SARS-CoV-2 in patients.

### Real-Time PCR Preparation

Table 5 demonstrates a reaction setup for one-step Reverse-Transcriptase Real-time PCR run profile, showing the cycling parameters utilised to identify and differentiate non-replicating SARS-CoV-2 from other SARS-CoV strains.

The following occurs within a positive pressure environment.
1. Thaw, vortex, and centrifuge the reaction components.
2. Add all the reaction components except the viral RNA sample to a tube as per the reaction setup table.
3. Multiply to accommodate the number of reactions required.
4. Vortex and centrifuge the mixture volume.
5. For a single reaction, add 19 μL of mixture to the real-time PCR tube.
6. Vortex and centrifuge the RNA template.
7. Add 6 μL of the RNA template to the PCR tube per reaction.
8. Apply the lids to the real-time PCR tubes and ensure that they are sealed.

### Real-Time PCR Machine Operation

1. Load the reaction tubes and fill the remaining slots with balance tube (containing water).
2. In the Samples table fill in:
   a. The Name column with sample identities.
   b. The Type column with the sample type (i.e. Standard, Control, NTC).
   c. The Assay column will autofill when selecting the sample type to match your assigned assay.
   d. If required, the Standards Concentration column is where you will input your concentrations. Unit of measurement can be selected at the top of the column.

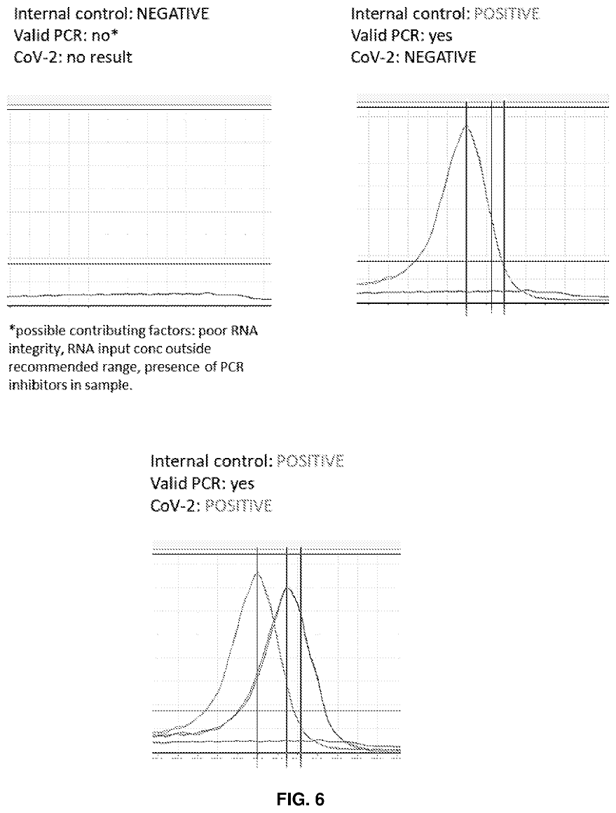

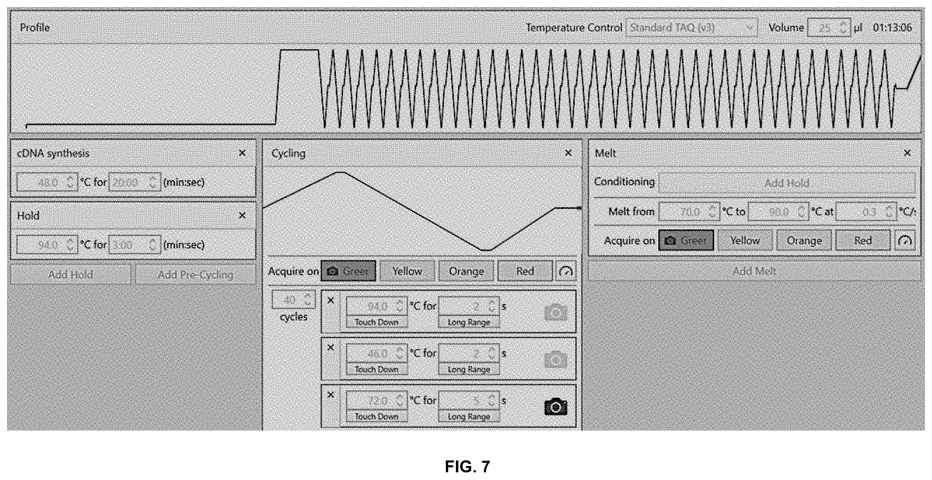

Once sample details have been provided, the PCR is ready for operation using the run profile conditions provided in FIG. 7 for the MIC platform Interpretation of RT-PCR results
1. Utilise the relevant Real-Time PCR machine's application for High-Resolution Melt curve analysis. Table 3 provides the melting temperatures of the amplified nucleic acid of SARS-CoV-2 and other SARS for the present test.
2. Utilise the relevant Real-Time PCR machine's application to determine the limit of detection of the assay based on a standard curve generated from serially diluted known concentration of reference gene target.

### Results

### Limit of Detection

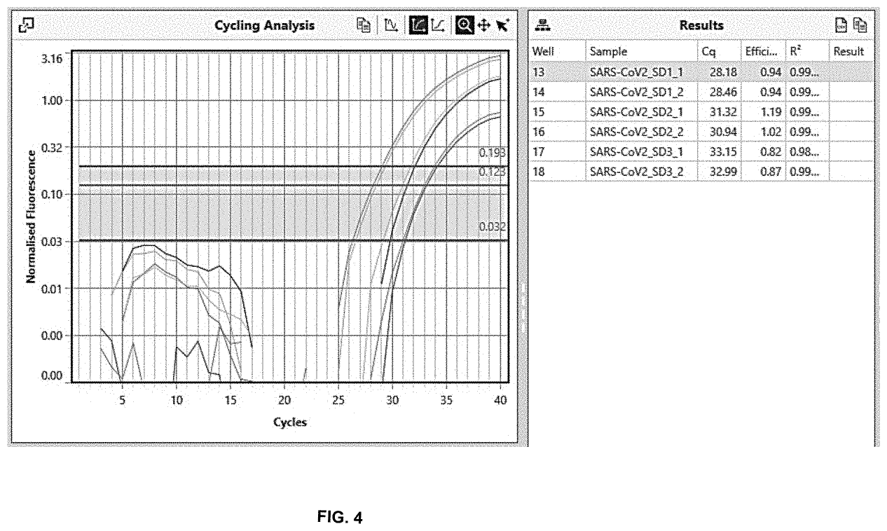

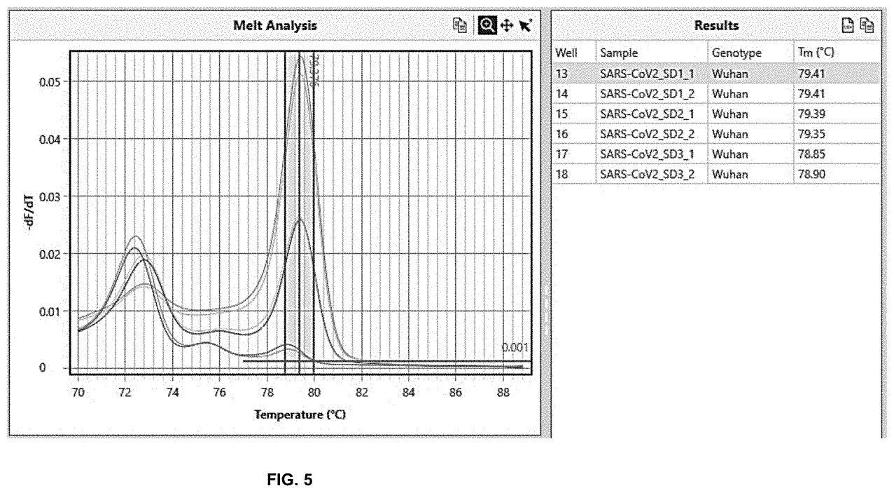

RT-PCR and HRM results for the above PCR test using a ten-fold serial dilution of SARS-CoV-2 RNA are shown in FIGS. 4 and 5 and Table 4. These results demonstrate that the test can detect as little as 5×10⁴ ng of viral RNA per mL, which is more sensitive that the typical viral load for nasal swabs and more sensitive than the typical viral load in COVID patient lungs.

### Interpreting Test Results

Exemplary results for the above PCR test for patients determined to be either negative or positive for SARS-CoV-2 are shown in FIG. 6.

TABLE 2
Primers and reaction parameters

| InfectID-CoV-2 assays | | | | | | | |
|---|---|---|---|---|---|---|---|
| | | | | | Amplicon | | Reaction |
| Primer | | | | | | Amplicon Tm | Optimal |
| | | Primer | Primer | | Amplicon- | (nearest | annealing |
| Primer | Seq 5′- | length | Tm | Target | length | neighbour) | temp |
| IC_RPP | CTTGGCTATTCAGTTG | 24 bp | 54.6° C. | RPP30 | 177 bp | 77.8° C. | 58.2° C. |
| 30-F | TTGCTATC (SEQ ID | | | | | | |
| | NO: 22) | | | | | | |
| IC_RPP | GTGAGATGGATCCGAG | 24 bp | 53.8° C. | RPP30 | | | |
| 30-R | ACAATAAT (SEQ ID | | | | | | |
| | NO: 23) | | | | | | |
| WUFor | GTGTTTTTAACATTTG | 22 bp | 47.5° C. | RdRp | 181 bp | 78.7° C. | 54.5° C. |
| | TCAAGC (SEQ ID | | | | | | |
| | NO: 24) | | | | | | |
| WuRev | TGTTACGCAAATATGCG | 28 bp | 45.9° C. | RdRp | | | |
| | (SEQ ID NO: 25) | | | | | | |

TABLE 3
Melting temperatures of SARS-CoV-2 and other SARS

| | Tm | |
|---|---|---|
| Sample | (° C.) | SARS Virus strain |
| 1 | 78.97 | Wuhan SARS-CoV-2 |
| 1 | 78.99 | Wuhan SARS-CoV-2 |
| 2 | 78.81 | Wuhan SARS-CoV-2 |
| 2 | 78.82 | Wuhan SARS-CoV-2 |
| 3 | 78.77 | Wuhan SARS-CoV-2 |
| 3 | 78.8 | Wuhan SARS-CoV-2 |
| 4 | 78.77 | Wuhan SARS-CoV-2 |
| 4 | 78.75 | Wuhan SARS-CoV-2 |
| 5 | 79.24 | Bat corona virus - China (Beta-CoV) |
| 5 | 79.28 | Bat corona virus - China (Beta-CoV) |
| 6 | 79.3 | SARS corona virus - Urbani strain - Asian |
| | | human SARS |
| 6 | 79.26 | SARS corona virus - Urbani strain - Asian |
| | | human SARS |
| 7 | 79.39 | SARS corona virus - mouse adapted coronavirus |
| 7 | 79.44 | SARS corona virus - mouse adapted coronavirus |
| 8 | 78.98 | SARS corona virus - clinical isolate CV7 |
| 8 | 79.03 | SARS corona virus - clinical isolate CV7 |
| 9 | 78.13 | SARS corona virus from Kenyan bats |
| 9 | 78.17 | SARS corona virus from Kenyan bats |
| 10 | 76.45 | SARS - Bat corona virus in European bats |
| 10 | 76.42 | SARS - Bat corona virus in European bats |

TABLE 4
Limit of detection results for SARS-CoV-2 RNA

| | | | | Calculated |
|---|---|---|---|---|
| | | | | Concentration |
| Well | Sample | Type | Cq | (ng/uL) |
| 5 | SD3-01 | Standard | 9.65 | 0.005 |
| 6 | SD3-02 | Standard | 9.62 | 0.005 |
| 7 | SD4-01 | Standard | 16.11 | 0.0005 |
| 8 | SD4-02 | Standard | 15.83 | 0.0005 |
| 9 | SD5-01 | Standard | 22.3 | 5.00E−05 |
| 10 | SD5-02 | Standard | 22.12 | 5.00E−05 |
| 11 | SD6-01 | Standard | 27.5 | 5.00E−06 |
| 12 | SD6-02 | Standard | 27.69 | 5.00E−06 |
| 13 | SARS-CoV2_SD1_1 | Virus RNA | 29.14 | 3.08E−06 |
| 14 | SARS-CoV2_SD1_2 | Virus RNA | 29.4 | 2.79E−06 |
| 15 | SARS-CoV2_SD2_1 | Virus RNA | 32.18 | 9.63E−07 |
| 16 | SARS-CoV2_SD2_2 | Virus RNA | 31.78 | 1.12E−06 |
| 17 | SARS-CoV2_SD3_1 | Virus RNA | 34.32 | 4.25E−07 |
| 18 | SARS-CoV2_SD3_2 | Virus RNA | 34.04 | 4.71E−07 |

*Limit of detection for non-replicating test = 0.0000004708 ng/uL

TABLE 5
Reaction Setup

| Components | Volume (μL) |
|---|---|
| Water | 0.75 |
| Source Template | 6 |
| Accumelt_sg Forward Primer | 2 |
| Accumelt_sg Reverse Primer | 2 |
| Accumelt (2×) | 12.5 |
| qScript XLT 1-step RT (25×) | 1 |
| MgCl2 | 0.75 |
| Total Volume (μL) | 25 |

TABLE 6
Additional primers for detection of inactive
SARS-CoV-2 strains

| Primer name | Sequence (5′ to 3′) |
|---|---|
| Forward 2 | GTGGCGGYTCACTHTATG |
| | (SEQ ID NO: 26) |
| Reverse 2 | GCTAGCYACTARACCTTG |
| | (SEQ ID NO: 27) |
| Reverse 3 | TGAGGTCCTTTAGTAAGG |
| | (SEQ ID NO: 28) |

*primers can be used as per the reaction setup in Table 4 and method described above

### Example 3

The methodology of Example 2 (referred to hereinafter as InfectID-Covid-19-D) can be employed on various RT-PCR platforms following the manufacturer's instructions. Samples tested include: cultured SARS-CoV-2 virus RNA; COVID patient RNA and synthetic oligonucleotide positive controls. These experiments demonstrate that the test is open-platform, and can be used on any real-time PCR platform that has melt-curve capability.

### Patient Samples Tested on the Bio-Rad CFX96 Real-Time PCR Machine.

Expected melt curve temperatures for InfectID-COVID-19-D testing on the Bio-Rad CFX96 real-time PCR machine.

| | | Melting |
|---|---|---|
| Assay | Target | temperature (tm) |
| InfectID-COVID-19-D | SARS-CoV-2 | 77.9° C. +/− 0.6° C. |
| (Detection) | RNA genome | |

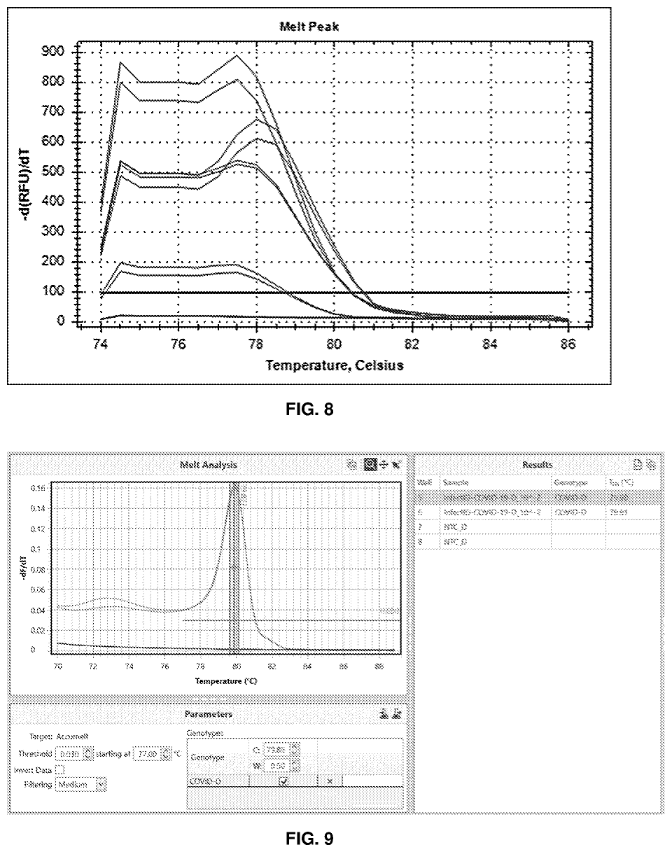

Experimental results (FIG. 8) show the correct melting temperature of 77.9° C. on the BioRad CFX96 machine, which is the expected melting temperature for the InfectID-COVID-19-D test.

Expected melt curve temperatures for InfectID-COVID-19-D testing on the MIC machine.

| | | MELTING |
|---|---|---|
| ASSAY | TARGET | TEMPERATURE (TM) |
| InfectID-COVID-19-D | SARS-CoV-2 | 79.2° C. +/− 1.0° C. |
| (Detection) | RNA genome | |

Experimental results (FIG. 9) show the correct melting temperature of 79.9° C. on the MIC machine, which is the expected melting temperature for the InfectID-COVID-19-D test.

Expected melt curve temperatures for InfectID-COVID-19-R testing on the QuantStudio 5 machine.

| | | MELTING |
|---|---|---|
| ASSAY | TARGET | TEMPERATURE (TM) |
| InfectID-COVID-19-D | SARS-CoV-2 | 78.6° C. +/− 0.6° C. |
| (Detection) | RNA genome | |

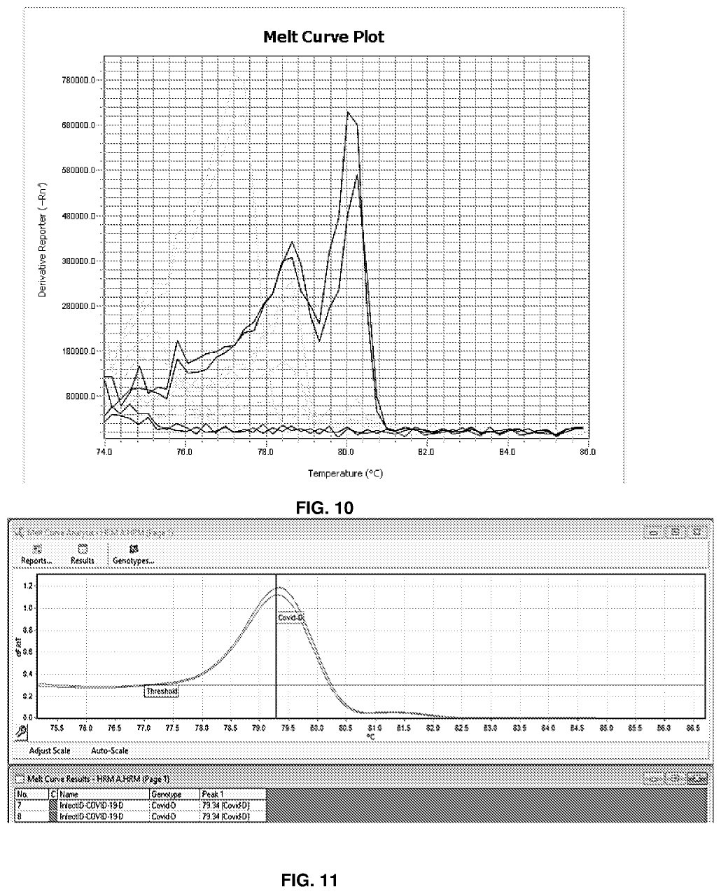

Experimental results (FIG. 10) show the correct melting temperature of 79.9° C. on the QuantStudio 5 machine, which is the expected melting temperature for the InfectID-COVID-19-D test.

Expected melt curve temperatures for InfectID-COVID-19-R testing on the RotorGeneQ machine.

| | | MELTING |
|---|---|---|
| ASSAY | TARGET | TEMPERATURE (TM) |
| InfectID-COVID-19-D | SARS-CoV-2 | 79.4° C. +/− 1.0° C. |
| (Detection) | RNA genome | |

Experimental results (FIG. 11) show the correct melting temperature of 79.34° C. on the RotorGeneQ machine, which is the expected melting temperature for the InfectID-COVID-19-D test.

The test of the present invention was validated for 105 SARS CoV 2 positive samples and 105 SARS CoV 2 Negative samples against CE IVD certified kits using kit comparison procedure under NABL ISO 15189-2012 guidelines strictly adhering to ICMR protocols. The assay was performed on a RotorgeneQ RT-PCR machine and a positive test showed the expected melting temperature set forth in Example 3 for that machine. The kit used for nucleic acid extraction were Qiagen-viral RNA Nucleic acid extraction. PCR was performed for extracted RNAs using RealStar SARS-CoV-2 rt-pcr Kit 1.0 for comparison.

Results were as follows:

TABLE 7

| InfectID- | RT-PCR test (RealStar *SARS-CoV-2 rt-pcr Kit) | | |
|---|---|---|---|
| COVID-19-D | Positive | Negative | Total |
| Positive | 100(TP) | 0(FP) | |
| Negative | 5(FN) | 105(TN) | |
| Total | 105 | 105 | 210 |

TP—True positive
TN—True negative
FP—False positive
FN—False Negative
Sensitivity: TP/(TP + FN) = 100/(100 + 5) = 95.23%
Specificity: TN/(TN + FP) = 105/(105 + 0) = 100%
Positive Predicative Value: TP/(TP + FP) = 100/(100 + 0) = 100%
Negative Predictive Value: TN/(TN + FN) = 105/105 + 5) = 95.45%

The test was also compared to applicant's validated test for identifying replication of covid-19 InfectID-R which is described in Australian patent application No. 202090624 filed on 27 Jul. 2020, the contents of which are incorporated herein by reference) and against RealStar*SARS-CoV-2 rt-pcr Kit 1.0 to trace the progression of the disease for 12 samples.

TABLE 8
Parallel evaluation of both the D and R kits

| | | | | | | | | | | | | |
|---|---|---|---|---|---|---|---|---|---|---|---|---|
| Kit | Day1 | Day2 | Day3 | Day4 | Day5 | Day6 | Day7 | Day8 | Day9 | Day10 | Day11 | Day12 |

Sample 1

| | | | | | | | | | | | | |
|---|---|---|---|---|---|---|---|---|---|---|---|---|
| InfectID | Pos | Pos | Pos | Pos | Pos | Neg | Neg | Neg | Neg | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 2

| | | | | | | | | | | | | |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Neg | Neg | Pos | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 3

| | | | | | | | | | | | | |
|---|---|---|---|---|---|---|---|---|---|---|---|---|
| InfectID | Pos | Pos | Pos | Pos | Pos | Pos | Neg | Pos | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 4

| | | | | | | | | | | | | |
|---|---|---|---|---|---|---|---|---|---|---|---|---|
| InfectID | Pos | Pos | Pos | Pos | Pos | INeg | Neg | Neg | Pos | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Neg | Neg | Neg | Neg | Neg | Neg |
| CoV-2 rt-pcr | | | | | | | | | | | | |
| Kit 1.0 | | | | | | | | | | | | |

Sample 5

| | | | | | | | | | | | | |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

Sample 6

| | | | | | | | | | | | | |
|---|---|---|---|---|---|---|---|---|---|---|---|---|
| InfectID | Pos | Pos | Pos | Pos | Pos | Pos | Pos | Pos | Neg | Neg | Neg | Neg |
| COVID-R | | | | | | | | | | | | |
| InfectID | Pos | Pos | Pos | Pos | Pos | Pos | Pos | Pos | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 7

| | | | | | | | | | | | | |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Neg | Neg | Neg | Neg | Neg | Neg |
| CoV-2 rt-pcr | | | | | | | | | | | | |
| Kit 1.0 | | | | | | | | | | | | |

Sample 8

| | | | | | | | | | | | | |
|---|---|---|---|---|---|---|---|---|---|---|---|---|
| InfectID | Pos | Pos | Pos | Pos | Pos | Pos | Pos | Pos | Pos | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | IPos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

Sample 9

| | | | | | | | | | | | | |
|---|---|---|---|---|---|---|---|---|---|---|---|---|
| InfectID | Pos | Pos | Pos | Pos | Pos | Pos | Neg | Pos | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Pos | Pos | Neg | Neg | Neg | Neg |
| CoV-2 rt-pcr | | | | | | | | | | | | |
| Kit 1.0 | | | | | | | | | | | | |

Sample 10

| | | | | | | | | | | | | |
|---|---|---|---|---|---|---|---|---|---|---|---|---|
| InfectID | Pos | Pos | Pos | Pos | Pos | Pos | Pos | Neg | Neg | Neg | Neg | Neg |
| COVID-R | | | | | | | | | | | | |
| InfectID | Pos | Pos | Pos | Pos | Pos | Pos | Pos | Neg | Neg | Neg | Neg | Neg |
| COVID-D | | | | | | | | | | | | |
| RealStar*SARS- | Pos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

Sample 11

| | | | | | | | | | | | | |
|---|---|---|---|---|---|---|---|---|---|---|---|---|
| InfectID | Pos | Pos | Pos | Pos | Pos | Pos | Neg | Pos | Neg | Neg | Neg | Neg |
| COVID-R | | | | | | | | | | | | |
| InfectID | Pos | Pos | Pos | Pos | Pos | Pos | Neg | Pos | Neg | Neg | Neg | Neg |
| COVID-D | | | | | | | | | | | | |
| RealStar*SARS- | Pos | Pos | Pos | Pos | Pos | Pos | Neg | Neg | Neg | Neg | Neg | Neg |
| CoV-2 rt-pcr | | | | | | | | | | | | |
| Kit 1.0 | | | | | | | | | | | | |

Sample 12

| | | | | | | | | | | | | |
|---|---|---|---|---|---|---|---|---|---|---|---|---|
| InfectID | Pos | Pos | Pos | Pos | Pos | Pos | Pos | Neg | Neg | Pos | Neg | Neg |
| COVID-R | | | | | | | | | | | | |
| InfectID | Pos | Pos | Pos | Pos | Pos | Pos | Pos | Neg | Neg | Pos | Neg | Neg |
| COVID-D | | | | | | | | | | | | |
| RealStar*SARS- | Pos | Pos | Pos | Pos | Pos | Pos | Pos | Neg | Neg | Neg | Neg | Neg |
| CoV-2 rt-pcr | | | | | | | | | | | | |
| Kit 1.0 | | | | | | | | | | | | |

The samples show that there is close concordance between the validated Realstar test and the assay described herein.

1. A method for detecting severe acute respiratory syndrome coronavirus 2 (SARS-CoV-2) in a sample, said method including the step of analysing at least a portion of a viral RNA-dependent RNA polymerase (RdRP) gene or gene product from the sample for the presence or absence of at least one single nucleotide polymorphism (SNP),
wherein the at least one SNP in the at least a portion of the viral RdRP gene or gene product is at a position corresponding to at least one of positions 137, 146, 272 and 380 of the nucleic acid sequence set forth in any one of SEQ ID NOs: 1-4;
wherein SARS-CoV-2 is detected in the sample based on the presence or absence of the at least one SNP.

2. The method of claim 1, wherein the at least one SNP in the at least a portion of the viral RdRP gene or gene product is at a position corresponding to at least one of positions 137 and 146 of the nucleic acid sequence set forth in any one of SEQ ID NOs: 1-4.

3. The method of claim 1, wherein the step of analysing comprises determining the presence or the absence of the at least one SNP using high resolution melt analysis, 5′ nuclease digestion, molecular beacons, oligonucleotide ligation, microarray, restriction fragment length polymorphism, antibody detection methods, direct sequencing or any combination thereof.

4. The method of claim 3, wherein the step of analysing comprises determining the presence or the absence of the at least one SNP using high resolution melt analysis.

5. The method of claim 1, wherein the step of analysing comprises amplifying a nucleic acid of the viral RdRP gene or gene product obtained from the sample.

6. The method of claim 5, wherein amplifying the nucleic acid of the viral RdRP gene or gene product utilizes reverse transcriptase-polymerase chain reaction (RT-PCR).

7. The method of claim 6, further including the steps of obtaining RNA from the sample and reverse transcribing the RNA to obtain cDNA.

8. The method of claim 5, wherein the step of amplifying the nucleic acid of the viral RdRP gene or gene product includes using a forward primer comprising, consisting of or consisting essentially of a nucleotide sequence set forth in SEQ ID NO: 24, a nucleotide sequence complementary thereto or a fragment or variant thereof and/or a reverse primer comprising, consisting of or consisting essentially of a nucleotide sequence set forth in SEQ ID NO: 25, SEQ ID NO: 27 or SEQ ID NO: 28, a nucleotide sequence complementary thereto or a fragment or variant thereof.

9.-16. (canceled)

17. The method of claim 1, wherein the sample is a biological sample, such as cells, blood, serum, plasma, saliva, cerebrospinal fluid, urine, stool, sputum, nasopharyngeal aspirates or swabs, obtained from a subject.

18. An isolated probe, tool or reagent capable of detecting SARS-CoV-2 in a sample, wherein the probe, tool or reagent is capable of binding, detecting or identifying the presence or absence of at least one SNP in at least a portion of a viral RdRP gene or gene product,
wherein the at least one SNP in the at least a portion of the viral RdRP gene or gene product is at a position corresponding to at least one of positions 137, 146, 272 and 380 of the nucleic acid sequence set forth in any one of SEQ ID NOs: 1-4.

19. The isolated probe, tool or reagent of claim 18, wherein the probe, tool or reagent comprises an oligonucleotide or primer comprising, consisting of or consisting essentially of a nucleic acid sequence as set forth in at least one of SEQ ID NOs: 24, 25, 27 and 28, a nucleotide sequence complementary thereto or a fragment or variant thereof.

20. An isolated oligonucleotide or primer comprising, consisting of or consisting essentially of a nucleic acid sequence as set forth in any one of SEQ ID NOs: 24, 25, 27 and 28, a nucleotide sequence complementary thereto or a fragment or variant thereof.

21. An array comprising: the isolated probe, tool or reagent of claim 18.

22. A biochip comprising a solid substrate and the isolated probe, tool or reagent of claim 18.

23. A kit or assay for detecting SARS-CoV-2 in a sample, said kit or assay comprising: the isolated probe, tool or reagent of claim 18.

24. The kit or assay of claim 23, comprising a pair of oligonucleotides, wherein at least one of the pair of oligonucleotides comprises, consists of or consists essentially of the nucleic acid sequence as set forth in any one of SEQ ID NOs: 24, 25, 27 and 28.

25. (canceled)

26. An array comprising the isolated oligonucleotide or primer of claim 20.

27. A biochip comprising the isolated oligonucleotide or primer of claim 20.

28. A kit or assay for detecting SARS-CoV-2 in a sample, said kit or assay comprising the isolated oligonucleotide or primer of claim 20.
